(19) United States
(12) Patent Application Publication
(10) Pub. No.: US 2022/0033881 A1
(43) Pub. Date: Feb. 3, 2022

(54) METHOD OF TESTING FOR SPECIFIC ORGANISMS IN AN INDIVIDUAL

(71) Applicant: Sabine Hazan, Ventura, CA (US)

(72) Inventor: Sabine Hazan, Ventura, CA (US)

(21) Appl. No.: 17/355,932

(22) Filed: Jun. 23, 2021

(30) Foreign Application Priority Data
Jul. 31, 2020 (WO) 2021026025

(51) Int. Cl. C12Q 1/689

(52) U.S. Cl. CPC C12Q 1/689

(57) ABSTRACT

A method of testing for specific organisms in an individual comprising the steps of: a) screening the individual; b) acquiring a stool sample from the individual; c) processing the stool sample to obtain the individual's microbiome; d) sequencing the microbiome of the individual; and e) analyzing the microbiome of the individual to determine whether one or more specific organisms are present in the individual, whereby a health condition of the individual is determined. The step of processing can comprise the sub-steps of: i) extracting DNA from the stool sample, which comprises adding the stool sample to a bead beating tube, achieving cell lysis, capturing the DNA on a silica membrane in a spin-column, and washing and eluting the DNA from the membrane; and ii) purifying the extracted DNA. A method of determining whether an individual has a health condition comprising the same steps. A stool sample collection kit.

### CROSS-REFERENCE TO RELATED APPLICATIONS

This application claims priority to PCT Application No. PCT/US2020/044605, titled “Method of Testing for Specific Organisms in an Individual,” filed Jul. 31, 2020, the contents of which are incorporated by reference herein in their entirety.

### BACKGROUND OF THE INVENTION

The human gastrointestinal (GI) microbiome is a complex, interconnected web of microbes, living in a symbiotic relationship with their host. There are greater than ten times more bacteria in our bodies than there are human cells, all in a delicate and ever-changing balance to maintain a healthy GI tract. When this balance is disrupted, a condition known as dysbiosis, or disease, can occur. There is still a debate over whether dysbiosis is a cause of disease or a symptom of it. Naturally, since the microbiome has such a profound impact on human health, including helping us digest food, make vitamins, and teach our immune cells to recognize pathogens, there is a desire study and learn as much about the microbiome as possible.

By correlating the microbiome data with survey data and medical records for the patients, connections may begin to be drawn between organisms present in the microbiome of the gastrointestinal tract, and a corresponding disease. For example, if there is one particular microbe in patients with Crohn's disease, the data suggest that this microbe could play a role in the cause or progression of this disease.

Accordingly, there is a need for a method of testing for specific organisms so that appropriate treatment may be rendered. The present invention satisfies this need.

### SUMMARY

In a first embodiment, the present invention is directed to my method of testing for specific organisms in an individual. The method comprises the steps of: a) screening the individual; b) acquiring a stool sample from the individual; c) processing the stool sample to obtain the individual's microbiome; d) sequencing the microbiome of the individual; and e) analyzing the microbiome of the individual to determine whether one or more specific organisms are present in the individual, whereby a health condition of the individual is determined.

The step of processing can comprise the sub-steps of: i) extracting DNA from the stool sample, which comprises adding the stool sample to a bead beating tube, achieving cell lysis, capturing the DNA on a silica membrane in a spin-column, and washing and eluting the DNA from the membrane; and ii) purifying the extracted DNA.

Optionally, step b) comprises providing the individual with a stool sample collection kit.

The stool sample collection kit can comprise a) at least one stool sample collection vial; b) at least one toilet accessory; c) at least one specimen bag; d) at least one pair of gloves; e) an authorization form; f) a patient information card; g) a questionnaire; and h) stool sample collection instructions.

Optionally, step b) comprises acquiring the stool sample from the individual via colonoscopy.

The one or more specific organisms of step e) can comprise one or more of the following: Acinetobacter baumannii, Actinomyces odontolyticus, Akkermansia muciniphila, Bacillus cereus, Bacillus subtilis, Bacteroides fragilis, Bacteroides vulgatus, Bifidobacterium adolescent, Blastocystis hominis, Butyrivibrio proteoclasticus, Campylobacter jejuni, Candida albicans, Chlamydophila pneumoniae, Clostridioides difficile, Clostridium beijerinckii, Clostridium perfringens, Clostridium sporgesse, Crptococcus neoformans, Cutibacterium acnes, Deinococcus radiodurans, Enterobacter cloacae, Enterococcus faecalis, Escherichia coli, Fusobacterium nucleatum, Helicobacter hepaticus, Helicobacter pylori, Klebsiella pneumoniae, Lactobacillus gasseri, Lactobacillus fermentum, Lactobacillus plantarum, Listeria monocytogenes, Mycobacterium avium subsp. paratuberculosis, Neisseria meningitides, Porphyromonas gingivalis, Proteus mirabilis, Pseudomonas aeruginosa, Rhodobacter sphaeroides, Saccharomyces cerevisiae, Salmonella enterica, Staphylococcus aureus, Staphylococcus epidermidis, Streptococcus agalactiae, Streptococcus mutano, Streptococcus pneumoniae, Streptococcus pyogenes, Toxoplasma gondii, Yersinia enterocolitica, and Bacteria X.

Optionally, step e) is an assay that tests for the following organisms: Acinetobacter baumannii, Actinomyces odontolyticus, Akkermansia muciniphila, Bacillus cereus, Bacillus subtilis, Bacteroides fragilis, Bacteroides vulgatus, Bifidobacterium adolescent, Blastocystis hominis, Butyrivibrio proteoclasticus, Campylobacter jejuni, Candida albicans, Chlamydophila pneumoniae, Clostridioides difficile, Clostridium beijerinckii, Clostridium perfringens, Clostridium sporgesse, Crptococcus neoformans, Cutibacterium acnes, Deinococcus radiodurans, Enterobacter cloacae, Enterococcus faecalis, Escherichia coli, Fusobacterium nucleatum, Helicobacter hepaticus, Helicobacter pylori, Klebsiella pneumoniae, Lactobacillus gasseri, Lactobacillus fermentum, Lactobacillus plantarum, Listeria monocytogenes, Mycobacterium avium subsp. paratuberculosis, Neisseria meningitides, Porphyromonas gingivalis, Proteus mirabilis, Pseudomonas aeruginosa, Rhodobacter sphaeroides, Saccharomyces cerevisiae, Salmonella enterica, Staphylococcus aureus, Staphylococcus epidermidis, Streptococcus agalactiae, Streptococcus mutano, Streptococcus pneumoniae, Streptococcus pyogenes, Toxoplasma gondii, Yersinia enterocolitica, and Bacteria X.

Optionally, step e) comprises comparing the microbiome of the individual to a microbiome of a mother of the individual.

Optionally, step e) comprises comparing the microbiome of the individual to a microbiome of a sibling of the individual.

Optionally, step e) comprises comparing the microbiome of the individual with a health condition to a microbiome of another individual with the same health condition.

Optionally, step e) comprises comparing the microbiome of the individual with a health condition to a microbiome of the individual before the individual had the health condition.

The method can further comprise step f) after step e), storing the processed stool sample in a freezer.

In a second embodiment, the present invention is directed to a method of determining whether an individual has a health condition. The method comprises the steps of: a) acquiring a stool sample from the individual; b) processing the stool sample to obtain the individual's microbiome; c) sequencing the microbiome of the individual; and d) analyzing the microbiome of the individual to determine whether one or more specific organisms are present in the individual, whereby the health condition of the individual is determined.

The health condition is selected from the group comprising: C. difficile infection, Obesity, Autism, Alzheimer's disease, Crohn's disease, Myalgic Encephalomyelitis/Chronic, Fatigue Syndrome (ME/CFS), Psoriasis, Chronic Urinary tract infection, Ulcerative Colitis, Multiple Sclerosis, Chronic constipation, Celiac sprue, Lyme disease, Elevated cholesterol, Colorectal cancer, Amyotrophic lateral sclerosis, Rheumatoid arthritis, Parkinson's disease, Depression, Anxiety, Obsessive-Compulsive disorder, Bipolar Disorder, Migraine headaches, Diabetes mellitus, Lupus, Epidermolysis, Metastatic mesothelioma, irritable bowel syndrome Diarrhea, irritable bowel syndrome Constipation, Eczema, Acne, Fatty liver, Myasthenia gravis, and Gout.

Step b) can comprise the steps of:
i) extracting DNA from the sample, which comprises the steps of adding the stool sample to a bead beating tube, achieving cell lysis, capturing DNA on a silica membrane in a spin-column, and washing and eluting the captured DNA from the membrane; and
ii) purifying the extracted DNA.

The one or more specific organisms of step d) can be selected from the group consisting of: Acinetobacter baumannii, Actinomyces odontolyticus, Akkermansia muciniphila, Bacillus cereus, Bacillus subtilis, Bacteroides fragilis, Bacteroides vulgatus, Bifidobacterium adolescent, Blastocystis hominis, Butyrivibrio proteoclasticus, Campylobacter jejuni, Candida albicans, Chlamydophila pneumoniae, Clostridioides difficile, Clostridium beijerinckii, Clostridium perfringens, Clostridium sporgesse, Crptococcus neoformans, Cutibacterium acnes, Deinococcus radiodurans, Enterobacter cloacae, Enterococcus faecalis, Escherichia coli, Fusobacterium nucleatum, Helicobacter hepaticus, Helicobacter pylori, Klebsiella pneumoniae, Lactobacillus gasseri, Lactobacillus fermentum, Lactobacillus plantarum, Listeria monocytogenes, Mycobacterium avium subsp. paratuberculosis, Neisseria meningitides, Porphyromonas gingivalis, Proteus mirabilis, Pseudomonas aeruginosa, Rhodobacter sphaeroides, Saccharomyces cerevisiae, Salmonella enterica, Staphylococcus aureus, Staphylococcus epidermidis, Streptococcus agalactiae, Streptococcus mutano, Streptococcus pneumoniae, Streptococcus pyogenes, Toxoplasma gondii, Yersinia enterocolitica, and Bacteria X.

### DRAWINGS

These and other features, aspects and advantages of the present invention will be better understood with reference to the following description, appended claims, and accompanying drawings where:

FIG. 1 is a flow chart of a method of testing an individual for specific organisms having features of the present invention;

FIG. 2 is a top plan view of a stool collection kit having features of the present invention;

FIG. 3 is top plan view of the stool collection kit of FIG. 2, wherein the contents have been removed from the box;

FIG. 4 is a graphical representation of the number of various mycobacterium found in the samples;

FIG. 5 is a graphical representation of the biodiversity of mycobacterium in healthy patients versus patients with Crohn's Disease of Example 1;

FIG. 6 is a graphical representation of the mycobacterium of patient 12 compared to patient 12's biological mother (patient 11) of Example 1;

FIG. 7 is a graphical representation of mycobacterium of patient 2 compared to patient 2's biological mother (patient 1) of Example 1;

FIG. 8 is a graphical representation of the mycobacterium of patient 10 versus patient 10's biological mother (patient 9) of Example 1;

FIG. 9 is a graphical representation of a comparison of the microbiome between patient 12 and patient 12's biological mother (patient 11) of Example 1;

FIG. 10 is a graphical representation of a comparison of the microbiome between patient 12 and patient 12's biological mother (patient 11) of Example 1;

FIG. 11 is a graphical representation of a comparison of the microbiome between patient 2 and patient 2's biological mother (patient 1) of Example 1;

FIG. 12 is a graphical representation of a comparison of the microbiome between patient 2 and patient 2's biological mother (patient 1) of Example 1;

FIG. 13 is a graphical representation of a comparison of the microbiome between patient 14 and patient 14's biological brother (patient 6) of Example 1;

FIG. 14 is a graphical representation of a comparison of the microbiome between patient 10 and patient 10's biological mother (patient 9) of Example 1;

FIG. 15 is a graphical representation showing common organisms found in patients with Crohn's disease of Example 1;

FIG. 16 is a graphical representation showing common organisms found in patients with Crohn's disease of Example 1;

FIG. 17 is a graphical representation showing common organisms found in patients with Crohn's disease of Example 1;

FIG. 18 is a graphical representation showing common organisms found in patients with Crohn's disease of Example 1;

FIG. 19 is a graphical representation showing common organisms found in patients with Crohn's disease of Example 1;

FIG. 20 is a graphical representation showing common organisms found in patients with Crohn's disease of Example 1;

FIG. 21 is a graphical representation showing common organisms found in patients with Crohn's disease of Example 1;

FIG. 22 is a graphical representation of a comparison of the microbiome between patient 1 and patient 1's biological mother of Example 1;

FIG. 23 is a graphical representation of a comparison of the microbiome between patient 12 and patient 12's biological mother (patient 11) of Example 1;

FIG. 24 is a graphical representation of a comparison of the microbiome between patient 2 and patient 2's biological mother of Example 1;

FIG. 25 is a graphical representation of a comparison of the microbiome between patient 14 and patient 14's biological brother of Example 1;

FIG. 26 is a graphical representation of a comparison of the microbiome between patient 12 and patient 12's biological mother of Example 1;

FIG. 27 is a graphical representation showing common organisms found in patients with Crohn's disease of Example 1;

FIG. 28 is a graphical representation showing common organisms found in patients with Crohn's disease of Example 1;

FIG. 29 is a graphical representation showing common organisms found in patients with Crohn's disease of Example 1;

FIG. 30 is a flow chart of a method of testing an individual that was infected with COVID-19 of Example 10; and

FIGS. 31A-31H are a series of graphs depicting whole genome alignment of SARS-CoV-2 in patients of Example 12.

### DETAILED DESCRIPTION OF THE INVENTION

The following discussion describes in detail one embodiment of the invention and several variations of that embodiment. This discussion should not be construed, however, as limiting the invention to those particular embodiments. Practitioners skilled in the art will recognize numerous other embodiments as well.

### Definitions

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

As used in this disclosure, the term “comprise” and variations of the term, such as “comprising” and “comprises,” are not intended to exclude other additives, components, integers, ingredients or steps.

### THE INVENTION

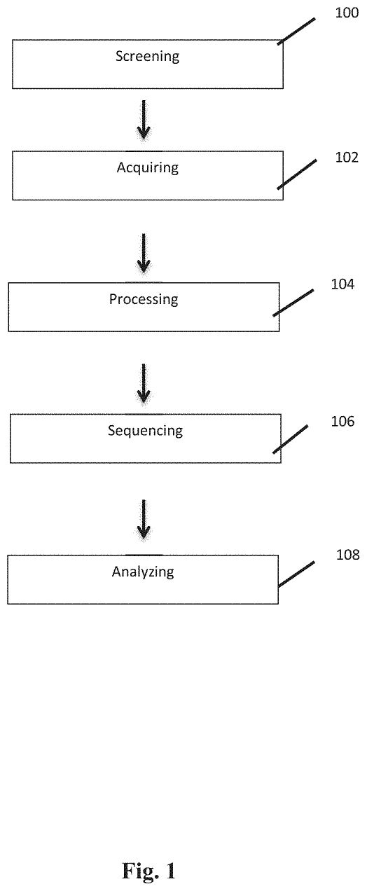

Referring now to FIG. 1, the present invention is a method of testing an individual for specific organisms. The method comprises five main steps: screening 100 the individual, acquiring 102 a stool sample from the individual, processing 104 the stool sample to obtain the individual's microbiome, sequencing 106 the microbiome of the individual, and analyzing 108 the microbiome of the individual to determine whether specific organisms are present in the individual.

During the step of screening, the individual typically undergoes the following: signing of the consent form, providing their medical history and demographics, having their vital signs taken/read, providing their height and weight, and providing the staff with a list of their prior and concomitant medications. Concomitant medications include any form of antibiotics, probiotics, or opiates.

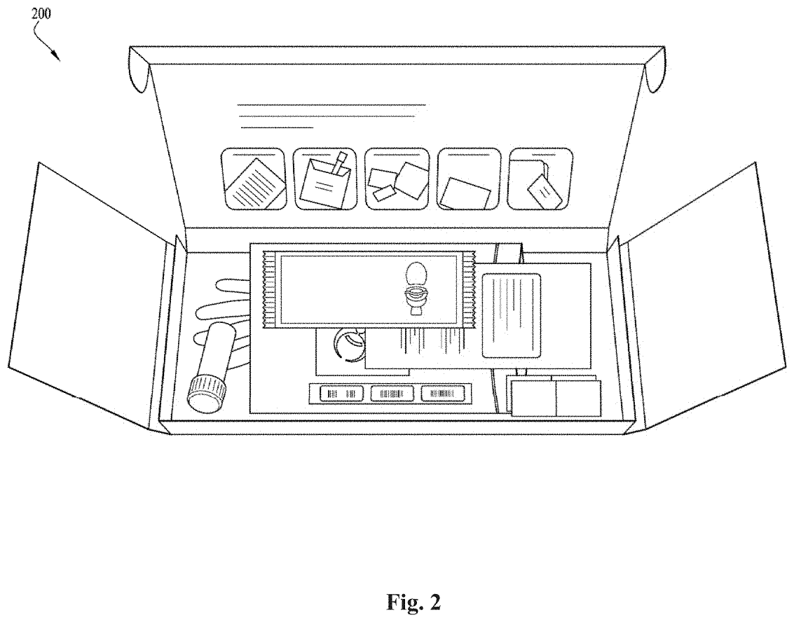

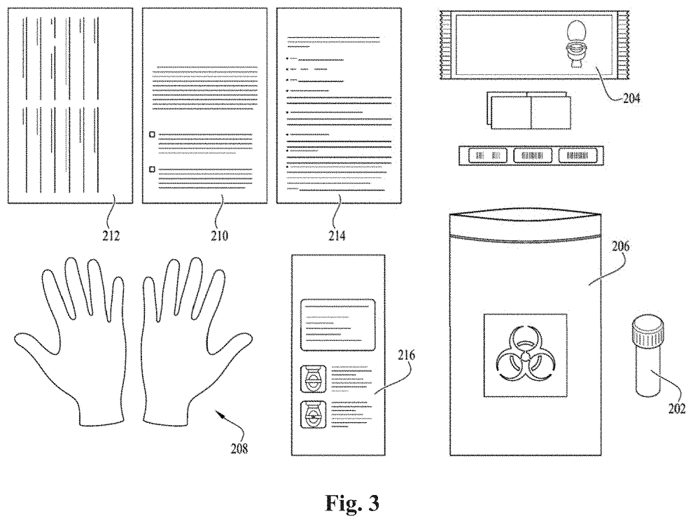

The individual then has a consultation to discuss the sequencing of their DNA and the method used to collect the fecal sample. For individual who collect their stool samples at home, they are provided with a stool collection kit 200 (shown in FIGS. 2 and 3) and instructed in their use. Individual who will have their stool sample collected via colonoscopy provided with colonoscopy preparation instructions and a prescription for bowel cleanse. As standard-of-care, a gastroenterologist will collect the colonoscopy samples during a medically necessary colonoscopy.

The individual then completes demographic and medical history forms to generate data to accompany their microbiome sequencing data.

As noted above, the step of acquiring a stool sample can either involve the stool sample collection kit 200 or a colonoscopy. The stool sample collection kit 200 is shown in FIGS. 2 and 3 and comprises: at least one stool sample collection vial 202, optionally the vial 200 contains a spoon, at least one toilet accessory or seat cover 204, at least one specimen bag 206, at least one pair of gloves 208, an authorization form 210, a patient information card 212, a questionnaire 214, and stool sample collection instructions 216.

The toilet accessory 204 is in the form of a circular strip of paper that slips over the toilet seat and creates a raised platform on which to provide the voided stool sample.

The stool sample collection instructions 216 are as follows: (1) Correctly position the toilet accessory (i.e. toilet cover) over the toilet seat and put on disposable latex gloves. (2) Unscrew the collection tube cap and use the spoon to scoop one spoonful of the stool sample from the feces. Do not pass the stool sample into the toilet or directly into the collection vial, and do not mix urine or water with the stool sample. (3) Place the stool sample into the collection vial. (4) Tighten the cap and shake to mix the contents thoroughly (and/or invert 10 times) to create a suspension. Some fecal material may be difficult to re-suspend. As long as the stool sample is suspended, the sample is stabilized. Foaming/frothing during shaking is normal. (5) Place the collection vial in the bag labeled “Specimen Bag-Biohazard” and seal the bag. (6) Place the bag back in the collection kit box. (7) Remove toilet cover and let it fall into the toilet bowl. Flush both the toilet cover and excess stool down the toilet. (8) Remove and dispose of gloves. Thoroughly wash hands.

Following collection of the stool sample, the stool sample is then processed and the microbiome analyzed. For these two steps, the following equipment is utilized: centrifuges, pipettes, thermocycler, fluorometers, vortexers, refrigerators/freezers, and a sequencing system (for example, an Illumina NextSeq 550 Sequencing System).

The step of processing the sample includes extracting and purifying patient DNA from the sample. Individual patient DNA is extracted and purified with a DNA extraction kit. Optionally, the QIAmp® PowerFecal® Pro DNA Kit can be used. The DNA extraction kit isolates both microbial and host genomic DNA from stool and gut samples.

In summary, for the DNA extraction step, the stool samples are added to a bead beating tube for rapid and thorough homogenization. Cell lysis occurs by mechanical and chemical methods. Total genomic DNA is captured on a silica membrane in a spin-column format. DNA is then washed and eluted from the membrane and ready for NGS, PCR and other downstream application.

Once the DNA has been extracted, the DNA is then quantitated using a fluorometer. The fluorometer can be a dual-channel fluorometer for nucleic acid quantitation. It provides highly sensitive fluorescent detection when quantifying nucleic acids and proteins.

The following steps are performed when quantitating the sample:

Mix 1-20 microliters of the extracted DNA sample and 200 microliters of dye in a 0.5 ml PCR tube. Mix well by pipetting or vortexing.

The fluorescence is then measured and the nucleic acid concentration is calculated and/or displayed.

Next, the library is prepared. The assay of the present invention is designed to detect all bacteria, viruses, and fungi that reside in the microbiome of the stool samples being evaluated. The assay utilizes an enzymatic reaction to fragment the DNA and to add adapter sequences. Library fabrication includes tagmentation, tagmentation clean-up, and an amplification step followed by another clean-up prior to pooling and sequencing.

The following definitions and abbreviations are used in this section:
BLT: Bead-Linked Transposomes
DNA: Deoxyribonucleic Acid
EPM: Enhanced PCR Mix
EtOH: Ethanol
NGS: Next Generation Sequencing
NTC: No Template Control
PCR: Polymerase Chain Reaction
RSB: Resuspension Buffer
SPB: Sample Purification Beads
TB1: Tagmentation Buffer
TSB: Tagment Stop Buffer
TWB: Tagment Wash Buffer

First, the BLT and TB1 are brought up to room temperature. Then, the BLT and TB1 are vortexed to mix.

Next, the appropriate volume of DNA is added to each well so that the total input amount is 100 nanograms. The optimal input for this assay is 100 nanograms, however, less DNA input can be utilized.

Next, the appropriate volume of nuclease-free water is added to the DNA samples to bring the total volume to 30 microliters.

Then, the BLT is vortexed vigorously for 10 seconds. Next, 11 microliters of BLT and 11 microliters of TB1 are combined for each sample, creating a tagmentation mastermix. Overage is included in this volume.

Next, the tagmentation master mix is vortexed and the volume is equally divided into an 8-tube strip.

Next, 20 microliters of the tagmentation master mix is transferred to each well containing a sample.

Then, the plate is sealed with Microseal ‘B’ and placed on a thermo cycler preprogrammed with the TAG program. The thermo cycler has a heated lid at 100° C. and reaction volume set to 50 microliters.

Next, the TAG program is run as shown in Table 1:

TABLE 1

| Cycle Step | Temperature | Time |
|---|---|---|
| Step 1 | 55° C. | 15 minutes |
| Step 2 | 10° C. | ∞ |

Once the TAG program is complete, the plate is removed from the thermo cycler.

Next, the Microseal ‘B’ seal is removed and 10 microliters of TSB is added to each sample.

Next, the plate is sealed with a Microseal ‘B’ and placed on the thermo cycler preprogrammed with the PTC program. The thermo cycler has a heated lid at 100° C.

Next, the PTC program is shown in Table 2:

TABLE 2

| Cycle Step | Temperature | Time |
|---|---|---|
| Step 1 | 37° C. | 15 minutes |
| Step 2 | 10° C. | ∞ |

When the PTC program is complete, the plate is removed from the thermo cycler and placed on a magnetic stand. The plate is left on the magnetic stand for about 3 minutes (as long as it takes for the solution to clear).

Once the solution is clear, the Microseal ‘B’ is removed from the plate and the supernatant is removed and discarded.

Next, the plate is removed from the magnetic stand and about 100 microliters of TWB is added. The sample should be pipetted slowly until the beads are fully re-suspended.

Next, the plate is placed back on the magnetic stand and approximately 3 more minutes pass while the solution clears again.

Once the solution clears, the supernatant is removed and discarded.

Next, the plate is removed from the magnetic stand and about 100 microliters of TWB is added. The sample should be pipetted slowly until the beads are fully re-suspended.

Next, the plate is again placed on the magnetic stand for an additional 3 minutes while the solution clears.

Next, 22 microliters of EPM and 22 microliters of nuclease-free water are combined with each sample to form a PCR mastermix. Overage is included in this volume. The PCR mastermix is vortexed and centrifuged.

With the plate on the magnetic stand, the supernatant is removed and discarded.

Next, the plate is removed from the magnetic stand and 40 microliters of PCR mastermix are immediately added directly onto the beads in each sample well.

The mastermix is immediately pipetted until the beads are fully re-suspended. Alternatively, the plate is sealed and a plate shaker is used at 1600 rpm for 1 minute.

Next, the plate is sealed with a Microseal ‘B’ and centrifuged at 280×g for 3 seconds.

Next, 10 microliters of index adapters are added to each sample in the plate. The plate is then centrifuged at 280×g for 30 seconds.

Next, the plate is placed on the thermo cycler that is preprogrammed with the BLT PCR program (and with lid preheated at 100° C.).

The BLT PCR Program is run as shown in Table 3:

TABLE 3

| Cycle Step | Number of Cycles | Temperature | Time | |
|---|---|---|---|---|
| Step 1 | 1 | 68° C. | 3 | minutes |
| Step 2 | 1 | 98° C. | 3 | minutes |
| Step 3 | 5 | 98° C. | 45 | seconds |
| | | 62° C. | 30 | seconds |
| | | 68° C. | 2 | minutes |
| Step 4 | 1 | 68° C. | 1 | minute |

| | | | |
|---|---|---|---|
| Step 5 | 1 | 10° C. | ∞ |

When BLT PCR program is complete, the plate is removed from the thermo cycler and centrifuged at 280×g for 1 minute.

Next, the plate is placed on the magnetic stand and it takes about 5 minutes for the solution to clear.

Next, about 45 microliters of supernatant are transferred from each well of the PCR plate to the corresponding well of a new midi plate.

Then, the midi plate is vortexed and the SPB is inverted multiple times to re-suspend.

Next, about 40 microliters of nuclease-free water is added to each sample well containing supernatant.

Next, about 45 microliters of SPB is added to each sample well. Each sample well is then mixed.

The plate is then sealed and incubated for 5 minutes at room temperature.

Next, the plate is placed on the magnetic stand and it takes about 5 minutes for the solution to clear.

Next, the SPB is vortexed thoroughly and 15 microliters of SPB is added to each well of a new midi plate.

Then, 125 microliters of supernatant is transferred from each well of the first plate into the corresponding well of the second midi plate containing 15 microliters SPB.

Each well of the second midi plate is then mixed and the first midi plate can be discarded.

The second midi plate is sealed and incubated for 5 minutes at room temperature.

The second midi plate is placed on the magnetic stand and it takes about 5 minutes for the solution to clear.

Next, without disturbing the beads, the supernatant is removed and discarded.

While the midi plate is still on the magnetic stand, 200 microliters of fresh 80% EtOH are added to the plate, without mixing. The plate is then incubated for 30 seconds.

Next, without disturbing the beads, the supernatant is removed and discarded.

While the second midi plate is still on the magnetic stand, about 200 microliters of fresh 80% EtOH are added, without mixing. The plate is then incubated for 30 seconds.

Next, without disturbing the beads, the supernatant is removed and discarded. Any residual EtOH is also removed and the second midi plate is allowed to air dry on the magnetic stand for about 5 minutes.

The second midi plate is removed from the magnetic stand and about 32 microliters of RSB is added to the beads.

The second midi plate is then re-suspended and incubated for about 2 minutes at room temperature.

The second midi plate is placed back on the magnetic stand it takes about 2 minutes for the solution to clear.

Once the solution clears, about 30 microliters of supernatant are transferred to a new 96-well PCR plate.

Next, the library is pooled and sequenced.

The following definitions and abbreviations are used in this section:
DNA: Deoxyribonucleic Acid
EtOH: Ethanol
HT1: Hybridization Buffer
NGS: Next Generation Sequencing
NTC: No Template Control
RSB: Resuspension Buffer
SAV: Sequencing Analysis Viewer

The following steps are taken to sequence the DNA:

Prepare the reagent cartridge for use.

Denature and dilute sample libraries.

Load pooled sample DNA libraries into the prepared reagent cartridge.

Set up and start the DNA sequencing using the selected DNA sequencing machine. The sequencing run can take approximately 27-30 hours to complete.

The bioinformatics pipeline utilizes a computational tool that profiles the microbial communities from metagenomic sequencing data with species level resolution. Patient microbiome profiles are analyzed to ascertain not only the profile of microbes in patient samples but also to identify specific strains, and provide accurate estimation of organismal abundance relative to the overall diversity.

Once the DNA is sequenced, the microbiome the individual patient is screened using the assay of the present invention, as noted above. The assay tests for the following organisms:
1. Acinetobacter baumannii
2. Actinomyces odontolyticus
3. Akkermansia muciniphila
4. Bacillus cereus
5. Bacillus subtilis
6. Bacteroides fragilis
7. Bacteroides vulgatus
8. Bifidobacterium adolescentis
9. Blastocystis hominis**(parasite)
10. Butyrivibrio proteoclasticus
11. Campylobacter jejuni
12. Candida albicans
13. Chlamydophila pneumoniae
14. Clostridioides difficile
15. Clostridium beijerinckii
16. Clostridium perfringens
17. Clostridium sporgesse
18. Crptococcus neoformans*(fungi)
19. Cutibacterium acnes
20. Deinococcus radiodurans
21. Enterobacter cloacae
22. Enterococcus faecalis
23. Escherichia coli
24. Fusobacterium nucleatum
25. Helicobacter hepaticus
26. Helicobacter pylori
27. Klebsiella pneumoniae
28. Lactobacillus gasseri
29. Lactobacillus fermentum
30. Lactobacillus plantarum
31. Listeria monocytogenes
32. Mycobacterium avium subsp. paratuberculosis
33. Neisseria meningitidis
34. Porphyromonas gingivalis
35. Proteus mirabilis
36. Pseudomonas aeruginosa
37. Rhodobacter sphaeroides
38. Saccharomyces cerevisiae*(fungi)
39. Salmonella enterica
40. Staphylococcus aureus
41. Staphylococcus epidermidis
42. Streptococcus agalactiae
43. Streptococcus mutano
44. Streptococcus pneumoniae
45. Streptococcus pyogenes
46. Toxoplasma gondii**(parasite)
47. Yersinia enterocolitica
48. Bacteria X

The step of analyzing the microbiome of the individual can include the following: comparing the microbiome of the individual to the microbiome of the individual's mother, comparing the microbiome of the individual to the microbiome of a sibling of the individual, comparing the microbiome of the individual with a health condition to the microbiome of another individual with same health condition, and comparing the microbiome of the individual with a health condition to the microbiome of the individual before they acquired the health condition (otherwise referred to as baseline versus non-baseline).

If the individual's baseline microbiome is being used in the analysis step, then the above recited steps of acquiring a stool sample, processing the stool sample, and sequencing the microbiome of the individual are performed at least twice—once before the individual acquires a health condition (known as a baseline) and at least once after the individual acquired the health condition. This is necessary so that the baseline microbiome can be compared to the microbiome when the individual is suffering from a health condition.

Optionally, the steps of acquiring a stool sample, processing the stool sample, and sequencing the microbiome of the individual are performed for a third time, after the individual has overcome the health condition, to confirm that the individual is healthy again.

When the assay shown above was tested on multiple individuals, the following organisms were detected as part of the assay: Bacteroides fragilis, Clostridioides difficile, Escherichia coli. The most abundant organism was Bacteroides fragilis (8.10%), and the mean abundance of the detected organisms was 2.87%. The total number of reads in the sample was 26,012,172.

Based upon phylum, the most abundant organisms were: Bacteroidetes at 80.90%, Firmicutes at 16.72%, Proteobacteria at 1.95%, Actinbacteria at 0.43%, Verrucomicrobia at 0.00%, Ascomycota at 0.00%, Candidatus Saccharibacteria at 0.00%, Fusobacteria at 0.00%, and Basidiomycota at 0.00%.

Based upon class, the most abundant organisms were: Bacteroidia at 80.90%, Clostridia at 15.49%, Betaproteobacteria at 0.99%, Deltaproteobacteria at 0.60%, Erysipelotrichia at 0.47%, Negativicutes at 0.41%, Gammaproteobacteria at 0.36%, Coriobacteria at 0.29%, Actinbacteria at 0.15%, and other at 0.35%.

Based upon family, the most abundant organisms were: Bacteriodaceae at 74.50%, Ruminococcaceae at 4.09%, Tannerellaceae at 3.32%, Rikenellaceae at 2.80%, Clostridiaceae at 2.12%, Lachnospiraceae at 1.99%, Eubacteriaceae at 1.83%, Sutterellaceae at 0.91%, Peptostreptococcaceae at 0.63% and other at 7.80%.

Based upon species, the most abundant organisms were: Bacteroides uniformis at 56.89%, Bacteroides fragilis at 8.10%, Bacteroides stercoris at 5.35%, Bacteroides stercoris CAG:120 at 4%, Clostridiales bacterium at between 4% and 3.3%, Parabacteriodes merdea at 3.32%, Faecalibacterium prausnitzil at 2.58%, Alistipes putredinis at 1.32%, [Eubacterium] hallii at 1.08%, and other at 13.78%

The present invention also comprises a screening kit or assay that screens for the above listed 48 organisms.

By screening for the above listed organisms, different diseases and conditions can be determined, such as: Autism, Crohn's disease, Chronic Urinary Tract Infections, Clostridoides difficile infection, Obesity, Alzheimer's disease, Psoriasis, Dietary Impact, Mylagic Encephalomyelitis/Chronic Fatigue Syndrome, the effect of diet, and COVID-19. See Appendix' B-M for the protocols related to these diseases/issues.

By applying the above procedures and screening for the 48 organisms listed above, it was determined that:

It is essential to compare the microbiomes of mother to child, sibling to sibling, and/or disease within disease;

Although everyone is an individual, each individual has a different microbiome;

A biological child of a mother is initially born with the same microbiome of the mother;

Within families of individuals, there is a similarity in the microbiome's between those familial individuals, however, people that are not related are not completely different;

Within diseases, there is a similarity in the microbiome of individuals that suffer from the same disease;

There is a loss of diversity of the microbiome in individuals with Crohn's disease and autism;

It is helpful to compare within the family or within the individual (baseline vs. disease, or disease vs. cured);

Toxoplasma gondii is a commonality found within patients with Crohn's Disease;

Loss of diversity was noted in children as compared to mothers;

In order for an individual to avoid getting Clostridium difficile, the individual needs multiple families of clostridiums within their gut. For an individual to avoid having the plague, the individual needs multiple families of Yersinia within their gut;

Clostridium difficile is present in everyone and Clostridium difficile generic testing is better than what is currently being utilized to test for Clostridium difficile;

Not all Crohn's Diseases are the same. There are different organisms that are involved that cause different versions of Crohn's Disease;

Obtaining a baseline from patients when they healthy and comparing that baseline to when they start developing a disease is important;

Sequencing the microbiome of a biological mother and a biological child, analyzing the differences between the two of them, and then comparing the differences between mother and child to other patients with the same disease showed that there was a difference in the organisms between the mother and child, and the microbiome varies from individual to individual. The child was then evaluated to determine what organisms the child was missing and the mother was then evaluated to determine what organisms the mother was missing, and the missing organisms from the mother and the child were then compared. It was noted that within families there is the same pattern of microbes (missing versus present); and High clostridiums bacteroides and staphylococcus are a marker of Celiac sprue;

Crohn's disease is multifactorial and can be caused by dysbiosis in the gut;

A high relative abundance of Akkermansia can cause neurological diseases;

A high relative abundance of Bacteroides vulgatus can cause anxiety; and

A loss of relative abundance of actinobacteria can cause loss of immunity.

### EXAMPLES

### Example 1: Crohn's Disease

Crohn's Disease (CD), a serious, potentially life-threatening, and debilitating condition which usually affects children, teenagers, and young adults, is an inflammatory bowel disease with a typical age of onset between 15 and 25 years of age. Symptoms can include pain, diarrhea, and other intestinal problems. CD appears to show some familial predisposition, as approximately 20-30% of people with CD have a direct blood relative with some form of IBD. Men and women are equally affected. The objective of this example is to determine the dysbiosis conditions under which Crohn's disease develops.

The following procedure was completed on 19 patients suffering from Crohn's disease. Shotgun Sequencing was performed. Shotgun sequencing is a laboratory technique for determining the DNA sequence of an organism's genome. The method involves breaking the genome into a collection of small DNA fragments that are sequenced individually. A computer program looks for overlaps in the DNA sequences and uses them to place the individual fragments in their correct order to reconstitute the genome.

More specifically, patient stool samples were collected utilizing collection vials. Following fecal collection, individual patient DNA was extracted purified with a DNA extraction kit. The isolated DNA was then quantitated utilizing a fluorometer.

After DNA quantification, the DNA was normalized and the library was prepared. This process utilized the shotgun workflow wherein the samples underwent tagmentation, purification, amplification and indexing, followed by a final purification step.

Samples libraries were then normalized and combined to create a library pool which was quantified and appropriately diluted to the final loading concentration to be sequenced on the appropriate DNA sequencing system/machine.

Once the DNA sequencing was complete, the raw.bcl data was converted to FASTQ files. The FASTQfiles were then pushed through the bioinformatics metagenomics pipeline with patient specific endpoint readouts profiling each individual's unique microbiome.

More specifically, the bioinformatics pipeline utilized a computational tool that profiled the microbial communities from metagenomic sequencing data with species level resolution. Patient microbiome profiles were then analyzed to ascertain not only the profile of microbes in the patient samples but also to identify specific strains, and provide accurate estimation of organismal abundance relative to the overall diversity.

Additionally, patient specific microbiome profiles were aligned and compared to their medical records and other patient provided information for further analysis and interpretation.

The patient sample was stored for future use in a 20° C. freezer.

Table 4 documents organisms that were discovered in each of the 19 patient samples. The first row of Table 4 contains the Patient ID numbers, which are represented throughout the Figures and Tables.

TABLE 4

| | A | B | C | D | E | 1 | 2 | 3 | 4 | 5 | 6 | 7 | 8 | 9 | 10 | 11 | 12 | 13 | 14 | T |
|---|---|---|---|---|---|---|---|---|---|---|---|---|---|---|---|---|---|---|---|---|
| Organism Name | | | | | | | | | | | | | | | | | | | | |
| [Clostridium] | 1 | 1 | 1 | 1 | 1 | 1 | 1 | 1 | 1 | 1 | 1 | 1 | 1 | 1 | 1 | 1 | 1 | 1 | 1 | 19 |
| bolteae | | | | | | | | | | | | | | | | | | | | |
| [Clostridium] | 1 | 1 | 0 | 1 | 1 | 1 | 1 | 0 | 1 | 1 | 1 | 1 | 1 | 1 | 1 | 1 | 1 | 1 | 1 | 17 |
| scindens | | | | | | | | | | | | | | | | | | | | |
| [Clostridium] | 1 | 1 | 1 | 1 | 1 | 1 | 1 | 1 | 1 | 1 | 1 | 1 | 1 | 1 | 1 | 1 | 1 | 1 | 1 | 19 |
| saccharolyticum | | | | | | | | | | | | | | | | | | | | |
| [Clostridium] | 1 | 1 | 1 | 1 | 1 | 1 | 1 | 0 | 1 | 1 | 1 | 1 | 1 | 1 | 1 | 1 | 1 | 1 | 1 | 18 |
| sphenoides | | | | | | | | | | | | | | | | | | | | |
| [Clostridium] | 1 | 1 | 1 | 1 | 1 | 1 | 1 | 0 | 1 | 1 | 1 | 1 | 1 | 1 | 1 | 1 | 1 | 1 | 1 | 18 |
| cellulosi | | | | | | | | | | | | | | | | | | | | |
| Clostridiales | 1 | 0 | 1 | 1 | 1 | 1 | 1 | 0 | 1 | 1 | 1 | 1 | 1 | 1 | 1 | 1 | 1 | 1 | 1 | 17 |
| bacterium | | | | | | | | | | | | | | | | | | | | |
| CCNA10 | | | | | | | | | | | | | | | | | | | | |
| Clostridiales | 1 | 0 | 1 | 1 | 1 | 1 | 1 | 0 | 1 | 1 | 1 | 1 | 1 | 1 | 1 | 1 | 1 | 1 | 1 | 17 |
| bacterium | | | | | | | | | | | | | | | | | | | | |
| 70B-A | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| sporogenes | | | | | | | | | | | | | | | | | | | | |
| Clostridium | 1 | 1 | 0 | 1 | 1 | 1 | 1 | 0 | 1 | 1 | 1 | 1 | 1 | 1 | 1 | 1 | 1 | 1 | 1 | 17 |
| sp. SY8519 | | | | | | | | | | | | | | | | | | | | |
| Clostridium | 0 | 1 | 1 | 1 | 0 | 1 | 1 | 1 | 1 | 1 | 1 | 1 | 1 | 1 | 1 | 1 | 1 | 1 | 1 | 17 |
| botulinum 202F | | | | | | | | | | | | | | | | | | | | |
| Clostridium | 1 | 1 | 1 | 1 | 1 | 1 | 1 | 0 | 1 | 1 | 1 | 0 | 1 | 1 | 1 | 1 | 1 | 1 | 1 | 17 |
| botulinum B | | | | | | | | | | | | | | | | | | | | |
| Clostridium | 0 | 0 | 0 | 0 | 0 | 0 | 1 | 0 | 1 | 1 | 1 | 1 | 1 | 1 | 1 | 1 | 1 | 1 | 1 | 12 |
| botulinum | | | | | | | | | | | | | | | | | | | | |
| BKT015925 | | | | | | | | | | | | | | | | | | | | |
| Clostridium | 0 | 0 | 0 | 1 | 1 | 1 | 0 | 0 | 0 | 1 | 0 | 1 | 0 | 1 | 0 | 1 | 1 | 0 | 0 | 8 |
| botulinum A2 | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| botulinum A3 | | | | | | | | | | | | | | | | | | | | |
| Clostridium | 1 | 1 | 1 | 1 | 1 | 1 | 0 | 0 | 1 | 1 | 1 | 1 | 0 | 1 | 0 | 1 | 1 | 0 | 0 | 13 |
| botulinum B1 | | | | | | | | | | | | | | | | | | | | |
| Org. Name Cont. | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 0 | 0 | 16 |
| botulinum F | | | | | | | | | | | | | | | | | | | | |
| Clostridium | 1 | 1 | 1 | 1 | 1 | 1 | 0 | 1 | 1 | 1 | 1 | 1 | 1 | 1 | 1 | 1 | 1 | 0 | 1 | 17 |
| botulinum | | | | | | | | | | | | | | | | | | | | |
| H04402 | | | | | | | | | | | | | | | | | | | | |
| Clostridium | 0 | 1 | 1 | 1 | 1 | 0 | 0 | 0 | 1 | 1 | 0 | 1 | 1 | 0 | 0 | 1 | 1 | 1 | 0 | 11 |
| botulinum | | | | | | | | | | | | | | | | | | | | |
| CDC_1436 | | | | | | | | | | | | | | | | | | | | |
| Clostridium | 0 | 1 | 0 | 0 | 0 | 0 | 0 | 0 | 0 | 1 | 0 | 1 | 0 | 0 | 0 | 1 | 1 | 0 | 0 | 5 |
| botulinum E3 | | | | | | | | | | | | | | | | | | | | |
| Clostridium | 0 | 0 | 0 | 0 | 0 | 0 | 0 | 0 | 1 | 1 | 1 | 1 | 1 | 0 | 0 | 1 | 1 | 1 | 0 | 8 |
| botulinum | | | | | | | | | | | | | | | | | | | | |
| Prevot_594 | | | | | | | | | | | | | | | | | | | | |
| Clostridium | 1 | 1 | 1 | 1 | 1 | 1 | 1 | 1 | 1 | 1 | 0 | 1 | 1 | 1 | 1 | 1 | 1 | 1 | 1 | 18 |
| perfringens | | | | | | | | | | | | | | | | | | | | |
| Clostridium | 0 | 1 | 1 | 1 | 1 | 1 | 0 | 0 | 1 | 1 | 1 | 1 | 1 | 1 | 0 | 1 | 1 | 0 | 1 | 14 |
| Perfringens F262 | | | | | | | | | | | | | | | | | | | | |
| Clostridium | 0 | 1 | 1 | 1 | 1 | 1 | 0 | 0 | 1 | 1 | 1 | 1 | 1 | 1 | 0 | 1 | 1 | 1 | 0 | 14 |
| perfringens | | | | | | | | | | | | | | | | | | | | |
| ATCC | | | | | | | | | | | | | | | | | | | | |
| Clostridium | 0 | 0 | 0 | 0 | 0 | 1 | 1 | 0 | 1 | 1 | 1 | 1 | 1 | 1 | 1 | 1 | 1 | 0 | 0 | 11 |
| perfringens | | | | | | | | | | | | | | | | | | | | |
| str. 13 | | | | | | | | | | | | | | | | | | | | |
| Clostridium | 0 | 1 | 1 | 0 | 1 | 1 | 1 | 1 | 1 | 1 | 1 | 1 | 1 | 1 | 1 | 1 | 1 | 1 | 1 | 17 |
| beijerinckii | | | | | | | | | | | | | | | | | | | | |
| Clostridium | 1 | 0 | 1 | 1 | 1 | 1 | 1 | 0 | 1 | 1 | 1 | 1 | 1 | 1 | 1 | 1 | 1 | 1 | 1 | 17 |
| beijerinckii | | | | | | | | | | | | | | | | | | | | |
| NCIMB | | | | | | | | | | | | | | | | | | | | |
| Clostridium | 1 | 0 | 0 | 1 | 0 | 1 | 1 | 0 | 1 | 1 | 1 | 1 | 1 | 1 | 1 | 1 | 1 | 1 | 1 | 15 |
| beijerinckii | | | | | | | | | | | | | | | | | | | | |
| NRRL | | | | | | | | | | | | | | | | | | | | |
| Clostridium | 1 | 1 | 1 | 1 | 1 | 1 | 1 | 1 | 1 | 1 | 1 | 1 | 1 | 1 | 1 | 1 | 1 | 1 | 1 | 19 |
| butyricum | | | | | | | | | | | | | | | | | | | | |
| Clostridium | 0 | 1 | 1 | 0 | 1 | 1 | 1 | 1 | 1 | 1 | 1 | 1 | 1 | 1 | 1 | 1 | 1 | 1 | 1 | 17 |
| baratii | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| sp. CT4 | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| pasteurianum | | | | | | | | | | | | | | | | | | | | |
| BC1 | | | | | | | | | | | | | | | | | | | | |
| Clostridium | 1 | 0 | 0 | 1 | 0 | 0 | 1 | 0 | 1 | 1 | 1 | 1 | 1 | 1 | 1 | 1 | 1 | 1 | 1 | 14 |
| baratii str. | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| isatidis | | | | | | | | | | | | | | | | | | | | |
| Clostridium | 1 | 0 | 1 | 0 | 1 | 1 | 1 | 0 | 1 | 1 | 1 | 1 | 1 | 1 | 1 | 1 | 1 | 1 | 1 | 16 |
| acetobutylicum | | | | | | | | | | | | | | | | | | | | |
| Clostridium | 1 | 1 | 0 | 0 | 1 | 0 | 0 | 0 | 0 | 1 | 1 | 0 | 0 | 0 | 0 | 0 | 1 | 0 | 0 | 6 |
| kluyveri | | | | | | | | | | | | | | | | | | | | |
| DSM 555 | | | | | | | | | | | | | | | | | | | | |
| Clostridium | 1 | 1 | 0 | 1 | 1 | 1 | 0 | 1 | 1 | 1 | 1 | 1 | 1 | 1 | 1 | 1 | 1 | 1 | 1 | 17 |
| sp. DL-VIII | | | | | | | | | | | | | | | | | | | | |
| Clostridium | 1 | 1 | 1 | 1 | 1 | 1 | 1 | 1 | 1 | 1 | 1 | 0 | 1 | 1 | 1 | 1 | 1 | 1 | 1 | 18 |
| aceticum | | | | | | | | | | | | | | | | | | | | |
| Clostridium | 1 | 1 | 1 | 1 | 1 | 1 | 1 | 1 | 1 | 1 | 1 | 1 | 1 | 1 | 1 | 1 | 1 | 1 | 1 | 19 |
| septicum | | | | | | | | | | | | | | | | | | | | |
| Clostridium | 1 | 0 | 0 | 1 | 0 | 1 | 1 | 0 | 1 | 1 | 1 | 1 | 1 | 1 | 1 | 1 | 1 | 1 | 1 | 15 |
| novyi NT | | | | | | | | | | | | | | | | | | | | |
| Clostridium | 1 | 1 | 1 | 0 | 1 | 0 | 0 | 0 | 0 | 1 | 1 | 1 | 1 | 1 | 1 | 1 | 1 | 1 | 1 | 14 |
| cellulovorans | | | | | | | | | | | | | | | | | | | | |
| 743B | | | | | | | | | | | | | | | | | | | | |
| Clostridium | 1 | 1 | 1 | 1 | 1 | 0 | 1 | 1 | 1 | 1 | 1 | 1 | 1 | 1 | 1 | 1 | 1 | 1 | 1 | 18 |
| argentinense | | | | | | | | | | | | | | | | | | | | |
| Clostridium | 1 | 1 | 1 | 1 | 1 | 1 | 0 | 0 | 1 | 1 | 1 | 1 | 1 | 1 | 1 | 1 | 1 | 1 | 1 | 17 |
| bornimense | | | | | | | | | | | | | | | | | | | | |
| Clostridium | 1 | 1 | 1 | 1 | 1 | 1 | 1 | 1 | 1 | 1 | 1 | 1 | 1 | 1 | 1 | 1 | 1 | 1 | 1 | 19 |
| sp. BNL1100 | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| cochlearium | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| sp. JN500901 | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| sp. JN-9 | | | | | | | | | | | | | | | | | | | | |
| Clostridium | 1 | 1 | 0 | 1 | 1 | 1 | 0 | 0 | 1 | 1 | 1 | 0 | 1 | 1 | 1 | 1 | 1 | 1 | 1 | 15 |
| sp. JN-1 | | | | | | | | | | | | | | | | | | | | |
| Clostridium | 1 | 1 | 1 | 1 | 1 | 1 | 0 | 1 | 0 | 1 | 1 | 1 | 1 | 1 | 1 | 1 | 1 | 1 | 1 | 17 |
| Saccharobutylicum | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| tyrobutyricum | | | | | | | | | | | | | | | | | | | | |
| Clostridium | 1 | 1 | 1 | 1 | 1 | 1 | 1 | 1 | 1 | 1 | 1 | 1 | 1 | 1 | 1 | 1 | 1 | 1 | 1 | 19 |
| estertheticum | | | | | | | | | | | | | | | | | | | | |
| Clostridium | 1 | 1 | 1 | 1 | 1 | 1 | 1 | 0 | 1 | 1 | 1 | 0 | 1 | 1 | 1 | 1 | 1 | 1 | 1 | 17 |
| Carboxidivorans | | | | | | | | | | | | | | | | | | | | |
| P7 | | | | | | | | | | | | | | | | | | | | |
| Clostridium | 1 | 0 | 1 | 1 | 1 | 1 | 1 | 1 | 1 | 1 | 1 | 1 | 1 | 1 | 1 | 1 | 1 | 1 | 1 | 18 |
| formicaceticum | | | | | | | | | | | | | | | | | | | | |
| Clostridium | 1 | 1 | 1 | 0 | 1 | 1 | 1 | 1 | 1 | 1 | 1 | 1 | 1 | 1 | 1 | 1 | 1 | 1 | 1 | 18 |
| chauvoei | | | | | | | | | | | | | | | | | | | | |
| Clostridium | 1 | 0 | 0 | 1 | 1 | 1 | 1 | 1 | 1 | 1 | 0 | 1 | 1 | 1 | 0 | 1 | 1 | 1 | 1 | 15 |
| sp. AWRP | | | | | | | | | | | | | | | | | | | | |
| Clostridium | 1 | 1 | 1 | 1 | 1 | 1 | 1 | 1 | 1 | 1 | 1 | 1 | 1 | 1 | 1 | 1 | 1 | 1 | 1 | 19 |
| tetani | | | | | | | | | | | | | | | | | | | | |
| Clostridium | 1 | 1 | 1 | 1 | 1 | 1 | 1 | 0 | 1 | 1 | 1 | 1 | 1 | 1 | 0 | 1 | 1 | 1 | 1 | 17 |
| tetani | | | | | | | | | | | | | | | | | | | | |
| 12124569 | | | | | | | | | | | | | | | | | | | | |
| Clostridium | 1 | 1 | 1 | 1 | 1 | 1 | 1 | 0 | 1 | 1 | 1 | 1 | 1 | 1 | 0 | 1 | 1 | 0 | 1 | 16 |
| tetani E88 | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| scatologenes | | | | | | | | | | | | | | | | | | | | |
| Clostridium | 1 | 0 | 1 | 1 | 1 | 1 | 0 | 0 | 1 | 1 | 1 | 1 | 1 | 1 | 1 | 1 | 1 | 1 | 1 | 16 |
| taeniosporum | | | | | | | | | | | | | | | | | | | | |
| Clostridium | 1 | 1 | 1 | 1 | 1 | 1 | 1 | 1 | 1 | 1 | 1 | 1 | 1 | 1 | 1 | 0 | 1 | 1 | 1 | 18 |
| drakei | | | | | | | | | | | | | | | | | | | | |
| Clostridium | 1 | 1 | 1 | 1 | 1 | 1 | 1 | 0 | 1 | 1 | 1 | 1 | 1 | 1 | 1 | 1 | 1 | 1 | 1 | 18 |
| autoethanogenum | | | | | | | | | | | | | | | | | | | | |
| Clostridium | 1 | 0 | 0 | 0 | 1 | 1 | 0 | 0 | 1 | 1 | 1 | 1 | 1 | 1 | 1 | 1 | 1 | 1 | 1 | 14 |
| sp. MF28 | | | | | | | | | | | | | | | | | | | | |
| Clostridium | 1 | 1 | 1 | 1 | 1 | 1 | 1 | 0 | 1 | 1 | 1 | 1 | 1 | 1 | 0 | 1 | 1 | 0 | 1 | 16 |
| ljungdahlii | | | | | | | | | | | | | | | | | | | | |
| DSM | | | | | | | | | | | | | | | | | | | | |
| Clostridiaceae | 1 | 1 | 1 | 1 | 1 | 1 | 1 | 0 | 0 | 1 | 1 | 1 | 1 | 1 | 1 | 1 | 1 | 1 | 1 | 17 |
| bacterium | | | | | | | | | | | | | | | | | | | | |
| 14S0207 | | | | | | | | | | | | | | | | | | | | |
| Clostridioides | 1 | 1 | 1 | 1 | 1 | 1 | 1 | 1 | 1 | 1 | 1 | 1 | 1 | 1 | 1 | 1 | 1 | 1 | 1 | 19 |
| difficile | | | | | | | | | | | | | | | | | | | | |
| Clostridioides | 1 | 1 | 1 | 1 | 1 | 1 | 1 | 0 | 1 | 1 | 1 | 1 | 1 | 1 | 1 | 1 | 1 | 1 | 1 | 18 |
| difficile ATCC | | | | | | | | | | | | | | | | | | | | |
| Clostridioides | 0 | 0 | 0 | 0 | 0 | 1 | 1 | 0 | 1 | 1 | 1 | 1 | 1 | 1 | 1 | 1 | 1 | 1 | 1 | 13 |
| difficile | | | | | | | | | | | | | | | | | | | | |
| QCD-63q42 | | | | | | | | | | | | | | | | | | | | |
| Clostridioides | 1 | 1 | 0 | 1 | 1 | 1 | 0 | 0 | 1 | 1 | 0 | 1 | 0 | 1 | 0 | 0 | 1 | 1 | 0 | 11 |
| difficile M120 | | | | | | | | | | | | | | | | | | | | |
| Clostridioides | 0 | 0 | 0 | 0 | 0 | 1 | 1 | 0 | 1 | 0 | 1 | 1 | 0 | 1 | 0 | 1 | 1 | 0 | 1 | 9 |
| difficile | | | | | | | | | | | | | | | | | | | | |
| QCD-37x79 | | | | | | | | | | | | | | | | | | | | |
| Clostridioides | 0 | 1 | 1 | 0 | 1 | 0 | 0 | 0 | 0 | 1 | 1 | 1 | 0 | 1 | 0 | 0 | 0 | 0 | 0 | 7 |
| difficile M68 | | | | | | | | | | | | | | | | | | | | |
| Clostridioides | 0 | 0 | 1 | 1 | 0 | 0 | 0 | 0 | 0 | 0 | 1 | 0 | 1 | 0 | 0 | 0 | 0 | 0 | 1 | 5 |
| difficile 630 | | | | | | | | | | | | | | | | | | | | |
| Clostridioides | 1 | 1 | 0 | 0 | 1 | 0 | 0 | 0 | 1 | 1 | 0 | 1 | 0 | 0 | 0 | 0 | 1 | 0 | 0 | 7 |
| difficile CIP | | | | | | | | | | | | | | | | | | | | |
| Clostridioides | 0 | 0 | 0 | 0 | 1 | 0 | 0 | 0 | 1 | 0 | 1 | 0 | 0 | 0 | 0 | 0 | 0 | 0 | 0 | 3 |
| difficile CD196 | | | | | | | | | | | | | | | | | | | | |
| Clostridioides | 0 | 0 | 0 | 0 | 0 | 0 | 0 | 0 | 0 | 1 | 0 | 0 | 0 | 0 | 0 | 1 | 1 | 0 | 0 | 3 |
| difficile | | | | | | | | | | | | | | | | | | | | |
| QCD-76w55 | | | | | | | | | | | | | | | | | | | | |
| Clostridioides | 0 | 0 | 0 | 0 | 0 | 0 | 0 | 0 | 0 | 0 | 0 | 0 | 1 | 0 | 0 | 0 | 1 | 0 | 1 | 3 |
| difficile | | | | | | | | | | | | | | | | | | | | |
| QCD-66c26 | | | | | | | | | | | | | | | | | | | | |
| [Clostridium] | 1 | 0 | 1 | 0 | 1 | 1 | 1 | 0 | 1 | 1 | 1 | 1 | 1 | 1 | 1 | 1 | 1 | 1 | 1 | 16 |
| innocuum | | | | | | | | | | | | | | | | | | | | |
| [Clostridium] | 1 | 0 | 1 | 0 | 1 | 1 | 1 | 0 | 0 | 1 | 1 | 1 | 1 | 1 | 1 | 1 | 1 | 1 | 1 | 15 |
| ultunense Esp | | | | | | | | | | | | | | | | | | | | |
| Clostridium | 0 | 1 | 0 | 0 | 0 | 0 | 0 | 0 | 0 | 0 | 0 | 0 | 0 | 0 | 0 | 0 | 0 | 0 | 0 | 1 |
| formicaceticum | | | | | | | | | | | | | | | | | | | | |
| Clostridium | 0 | 1 | 1 | 0 | 0 | 0 | 0 | 1 | 0 | 0 | 0 | 0 | 0 | 0 | 0 | 0 | 0 | 0 | 0 | 3 |
| sp. AWRP | | | | | | | | | | | | | | | | | | | | |
| Clostridiales | 0 | 1 | 0 | 0 | 0 | 0 | 0 | 1 | 0 | 0 | 0 | 0 | 0 | 0 | 0 | 0 | 0 | 1 | 0 | 3 |
| bacterium | | | | | | | | | | | | | | | | | | | | |
| CCNA10 | | | | | | | | | | | | | | | | | | | | |
| Clostridiales | 0 | 1 | 0 | 0 | 0 | 0 | 0 | 1 | 0 | 0 | 0 | 0 | 0 | 0 | 0 | 0 | 0 | 1 | 0 | 3 |
| bacterium | | | | | | | | | | | | | | | | | | | | |
| 70B-A | | | | | | | | | | | | | | | | | | | | |
| [Clostridium] | 0 | 1 | 0 | 1 | 0 | 1 | 0 | 0 | 1 | 0 | 0 | 0 | 0 | 0 | 0 | 0 | 0 | 1 | 1 | 6 |
| ultunense Esp | | | | | | | | | | | | | | | | | | | | |
| Clostridium | 0 | 0 | 1 | 1 | 0 | 0 | 1 | 1 | 1 | 1 | 0 | 1 | 1 | 1 | 1 | 1 | 1 | 1 | 1 | 14 |
| kluyveri | | | | | | | | | | | | | | | | | | | | |
| Clostridium | 0 | 0 | 0 | 1 | 0 | 1 | 1 | 1 | 1 | 0 | 0 | 0 | 1 | 0 | 0 | 0 | 0 | 1 | 0 | 7 |
| cellulovorans | | | | | | | | | | | | | | | | | | | | |
| 743B | | | | | | | | | | | | | | | | | | | | |
| Clostridium | 0 | 0 | 0 | 0 | 0 | 0 | 0 | 0 | 1 | 1 | 1 | 1 | 1 | 1 | 1 | 0 | 1 | 1 | 1 | 10 |
| Saccharoperbutylacetonicum | | | | | | | | | | | | | | | | | | | | |
| Total | 59 | 59 | 57 | 60 | 64 | 65 | 56 | 26 | 70 | 75 | 69 | 70 | 70 | 70 | 59 | 70 | 76 | 66 | 66 | |
| Clostridia | | | | | | | | | | | | | | | | | | | | |
| species | | | | | | | | | | | | | | | | | | | | |
| | | | | | | | | 26 | | 75 | | | | | 59 | | 76 | 66 | 66 | |

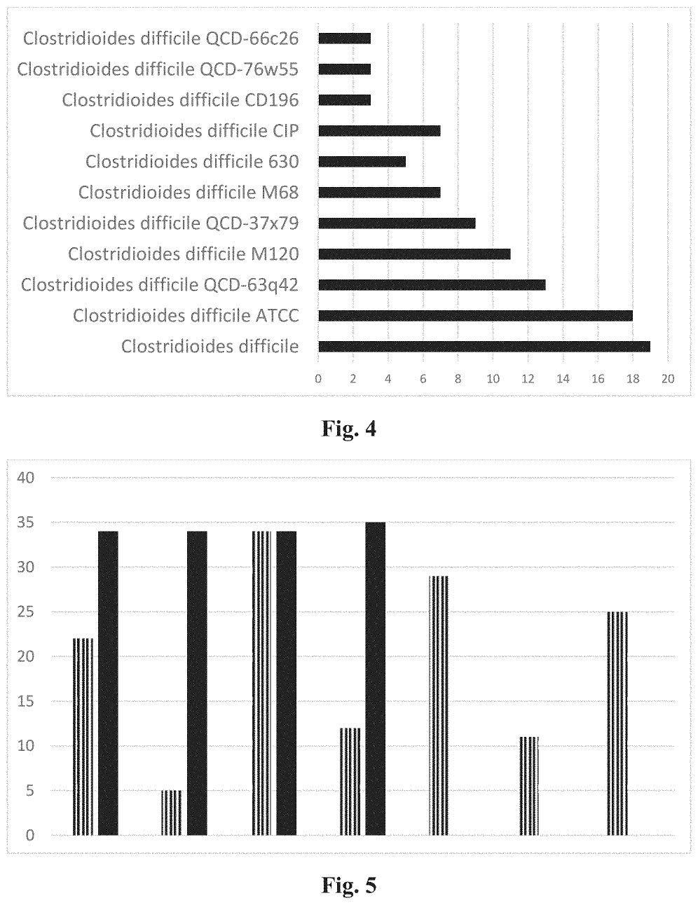

Table 5, shown below, documents the total numbers of the different species of bacteria/organisms present in all 19 patient samples combined. The data documented in Table 5 is shown in FIG. 4.

TABLE 5

| | Number Present |
|---|---|
| Species | |
| Clostridioides difficile | 19 |
| Clostridioides difficile ATCC | 18 |
| Clostridioides difficile QCD-63q42 | 13 |
| Clostridioides difficile M120 | 11 |
| Species Cont. | |
| Clostridioides difficile QCD-37x79 | 9 |
| Clostridioides difficile M68 | 7 |
| Clostridioides difficile 630 | 5 |
| Clostridioides difficile CIP | 7 |
| Clostridioides difficile CD196 | 3 |
| Clostridioides difficile QCD-76w55 | 3 |
| Clostridioides difficile QCD-66c26 | 3 |

Table 6 documents the mycobacterium found in the samples.

TABLE 6

| | 1 | 6 | 9 | 11 | 2 | 3 | 5 | 10 | 12 | 13 | 14 | Total | Organism name |
|---|---|---|---|---|---|---|---|---|---|---|---|---|---|
| Organism Name | | | | | | | | | | | | | |
| Mycobacterium | 1 | 1 | 1 | 1 | 1 | 0 | 1 | 1 | 1 | 0 | 1 | 5 | Salmonella enterica |
| colombiense | | | | | | | | | | | | | |
| Mycobacterium | 1 | 1 | 1 | 1 | 0 | 0 | 1 | 0 | 1 | 0 | 1 | 3 | Salmonella enterica |
| chimaera | | | | | | | | | | | | | subsp. enterica |
| | | | | | | | | | | | | | serovar Brancaster |
| Mycobacterium | 1 | 1 | 1 | 1 | 0 | 0 | 1 | 1 | 1 | 0 | 1 | 4 | Salmonella enterica |
| intracellulare | | | | | | | | | | | | | subsp. enterica |
| subsp. | | | | | | | | | | | | | serovar Chester |
| Yongonense | | | | | | | | | | | | | |
| Mycobacterium | 1 | 0 | 1 | 1 | 1 | 1 | 1 | 1 | 1 | 1 | 1 | 7 | |
| avium | | | | | | | | | | | | | |
| Mycobacterium | 1 | 0 | 1 | 1 | 0 | 1 | 1 | 0 | 0 | 0 | 0 | 2 | Salmonella enterica |
| avium subsp. | | | | | | | | | | | | | subsp. enterica |
| Paratuberculosis | | | | | | | | | | | | | serovar Minnesota |
| Mycobacterium | 0 | 1 | 1 | 1 | 1 | 0 | 1 | 0 | 0 | 0 | 1 | 3 | |
| avium subsp. | | | | | | | | | | | | | |
| hominissuis | | | | | | | | | | | | | |
| Mycobacterium | 0 | 1 | 0 | 1 | 0 | 0 | 1 | 0 | 0 | 0 | 0 | 1 | |
| avium 104 | | | | | | | | | | | | | |
| Org. Name Cont. | | | | | | | | | | | | | |
| Mycobacterium | 1 | 1 | 1 | 1 | 1 | 0 | 1 | 1 | 1 | 0 | 1 | 5 | Salmonella enterica |
| marseillense | | | | | | | | | | | | | subsp. enterica |
| | | | | | | | | | | | | | serovar Macclesfield |
| Mycobacterium | 1 | 1 | 1 | 1 | 0 | 0 | 1 | 0 | 1 | 0 | 1 | 3 | Salmonella enterica |
| lepraemurium | | | | | | | | | | | | | subsp. enterica |
| | | | | | | | | | | | | | serovar Tennessee |
| Mycobacterium | 1 | 1 | 1 | 1 | 0 | 0 | 1 | 0 | 0 | 0 | 0 | 1 | Salmonella enterica |
| paraintracellulare | | | | | | | | | | | | | subsp. enterica |
| | | | | | | | | | | | | | serovar Rubislaw |
| Mycobacterium | 1 | 1 | 1 | 1 | 1 | 0 | 1 | 0 | 1 | 1 | 1 | 5 | Salmonella enterica |
| sp. EPa45 | | | | | | | | | | | | | subsp. enterica |
| | | | | | | | | | | | | | serovar Typhimurium |
| Mycobacterium | 1 | 1 | 1 | 1 | 1 | 0 | 1 | 1 | 1 | 1 | 1 | 6 | Salmonella enterica |
| sp. YC-RL4 | | | | | | | | | | | | | subsp. enterica |
| | | | | | | | | | | | | | serovar Senftenberg |
| Mycobacterium | 1 | 1 | 1 | 1 | 1 | 0 | 1 | 1 | 1 | 1 | 1 | 6 | Salmonella enterica |
| sp. MS1601 | | | | | | | | | | | | | subsp. enterica |
| | | | | | | | | | | | | | serovar Waycross |
| Mycobacterium | 1 | 1 | 1 | 1 | 1 | 0 | 1 | 0 | 1 | 1 | 1 | 5 | Salmonella enterica |
| dioxanotrophicus | | | | | | | | | | | | | subsp. enterica |
| | | | | | | | | | | | | | serovar Weltevreden |
| Mycobacterium | 1 | 1 | 1 | 1 | 0 | 0 | 1 | 0 | 1 | 0 | 1 | 3 | Salmonella enterica |
| sp. VKM Ac- | | | | | | | | | | | | | subsp. enterica |
| 1817D | | | | | | | | | | | | | serovar Choleraesuis |
| Mycobacterium | 1 | 1 | 1 | 1 | 1 | 0 | 1 | 1 | 1 | 0 | 1 | 5 | Salmonella enterica |
| kansasii | | | | | | | | | | | | | subsp. enterica |
| | | | | | | | | | | | | | serovar Saintpaul |
| Mycobacterium | 1 | 1 | 1 | 1 | 1 | 0 | 1 | 0 | 1 | 1 | 1 | 5 | Salmonella enterica |
| sp. djl-10 | | | | | | | | | | | | | subsp. enterica |
| | | | | | | | | | | | | | serovar Stanley |
| Mycobacterium | 1 | 1 | 1 | 1 | 1 | 0 | 1 | 0 | 1 | 0 | 1 | 4 | Salmonella enterica |
| sp. JS623 | | | | | | | | | | | | | subsp. enterica |
| | | | | | | | | | | | | | serovar Apapa |
| Mycobacterium | 1 | 1 | 1 | 1 | 1 | 0 | 1 | 1 | 1 | 0 | 1 | 5 | Salmonella enterica |
| leprae | | | | | | | | | | | | | subsp. enterica |
| | | | | | | | | | | | | | serovar Djakarta |
| Mycobacterium | 1 | 1 | 0 | 1 | 1 | 1 | 1 | 0 | 1 | 0 | 0 | 4 | Salmonella enterica |
| shigaense | | | | | | | | | | | | | subsp. enterica |
| | | | | | | | | | | | | | serovar Albany |
| Mycobacterium | 1 | 1 | 1 | 1 | 1 | 1 | 1 | 1 | 1 | 1 | 1 | 7 | Salmonella enterica |
| sp. DL90 | | | | | | | | | | | | | subsp. enterica |
| | | | | | | | | | | | | | serovar Milwaukee |
| Mycobacterium | 1 | 1 | 1 | 1 | 0 | 0 | 1 | 0 | 0 | 0 | 0 | 1 | Salmonella enterica |
| canettii CIPT | | | | | | | | | | | | | subsp. enterica |
| 140070010 | | | | | | | | | | | | | serovar Thompson |
| Mycobacterium | 1 | 1 | 1 | 1 | 0 | 0 | 1 | 0 | 1 | 0 | 0 | 2 | Salmonella enterica |
| canettii CIPT | | | | | | | | | | | | | subsp. enterica |
| 140070017 | | | | | | | | | | | | | serovar Stanleyville |
| Mycobacterium | 0 | 0 | 0 | 0 | 0 | 0 | 1 | 0 | 1 | 0 | 0 | 2 | |
| canettii CIPT | | | | | | | | | | | | | |
| 140070008 | | | | | | | | | | | | | |
| Mycobacterium | 0 | 1 | 0 | 0 | 0 | 0 | 0 | 0 | 0 | 0 | 0 | 0 | |
| canettii CIPT | | | | | | | | | | | | | |
| 140010059 | | | | | | | | | | | | | |
| Mycobacterium | 1 | 1 | 1 | 1 | 1 | 0 | 1 | 0 | 1 | 1 | 1 | 5 | Salmonella enterica |
| tuberculosis | | | | | | | | | | | | | subsp. salamae |
| | | | | | | | | | | | | | serovar 55:k:z39 |
| Mycobacterium | 1 | 1 | 1 | 1 | 1 | 0 | 1 | 0 | 1 | 0 | 1 | 4 | |
| haemophilum | | | | | | | | | | | | | |
| Mycobacterium | 1 | 1 | 1 | 1 | 1 | 0 | 1 | 0 | 1 | 0 | 1 | 4 | |
| haemophilum | | | | | | | | | | | | | |
| DSM 44634 | | | | | | | | | | | | | |
| Mycobacterium | 1 | 1 | 1 | 1 | 1 | 0 | 1 | 1 | 1 | 0 | 1 | 5 | |
| sp. WY10 | | | | | | | | | | | | | |
| Mycobacterium | 1 | 1 | 1 | 1 | 0 | 1 | 1 | 0 | 1 | 1 | 1 | 5 | |
| paragordonae | | | | | | | | | | | | | |
| Mycobacterium | 1 | 1 | 1 | 1 | 1 | 0 | 1 | 0 | 1 | 1 | 1 | 5 | |
| marinum | | | | | | | | | | | | | |
| Mycobacterium | 1 | 1 | 1 | 1 | 1 | 0 | 1 | 1 | 1 | 1 | 0 | 5 | |
| sp. JLS | | | | | | | | | | | | | |
| Mycobacterium | 1 | 1 | 1 | 1 | 1 | 0 | 1 | 0 | 0 | 0 | 0 | 2 | |
| sp. PYR15 | | | | | | | | | | | | | |
| Mycobacterium | 1 | 1 | 1 | 0 | 0 | 0 | 1 | 0 | 1 | 0 | 0 | 2 | |
| ulcerans subsp. | | | | | | | | | | | | | |
| shinshuense | | | | | | | | | | | | | |
| Mycobacterium | 1 | 0 | 1 | 1 | 1 | 0 | 0 | 1 | 1 | 0 | 0 | 3 | |
| liflandii 128FXT | | | | | | | | | | | | | |
| Mycobacterium | 1 | 1 | 1 | 1 | 0 | 0 | 0 | 0 | 1 | 0 | 1 | 2 | |
| sp. QIA-37 | | | | | | | | | | | | | |
| [Mycobacterium] | 1 | 1 | 1 | 1 | 0 | 0 | 0 | 0 | 0 | 0 | 0 | 0 | |
| stephanolepidis | | | | | | | | | | | | | |
| [Mycobacterium] | 1 | 1 | 1 | 1 | 0 | 0 | 0 | 0 | 0 | 0 | 0 | 0 | |
| chelonae subsp. | | | | | | | | | | | | | |
| Gwanakae | | | | | | | | | | | | | |
| Mycobacterium | 0 | 0 | 0 | 0 | 0 | 0 | 1 | 0 | 0 | 0 | 0 | 1 | |
| sp. MOTT36Y | | | | | | | | | | | | | |
| Mycobacterium | 0 | 0 | 0 | 0 | 0 | 0 | 0 | 0 | 0 | 0 | 1 | 1 | |
| pseudoshottsii | | | | | | | | | | | | | |
| JCM 15466 | | | | | | | | | | | | | |
| | 34 | 34 | 34 | 35 | 22 | 5 | 34 | 12 | 29 | 11 | 25 | 19.7 | |

FIG. 5 is a graphical representation of the biodiversity of mycobacterium in healthy patients versus patients with Crohn's Disease. Crohn's patients are shown using the solid black bars and healthy patients are shown using the series of smaller black bars.

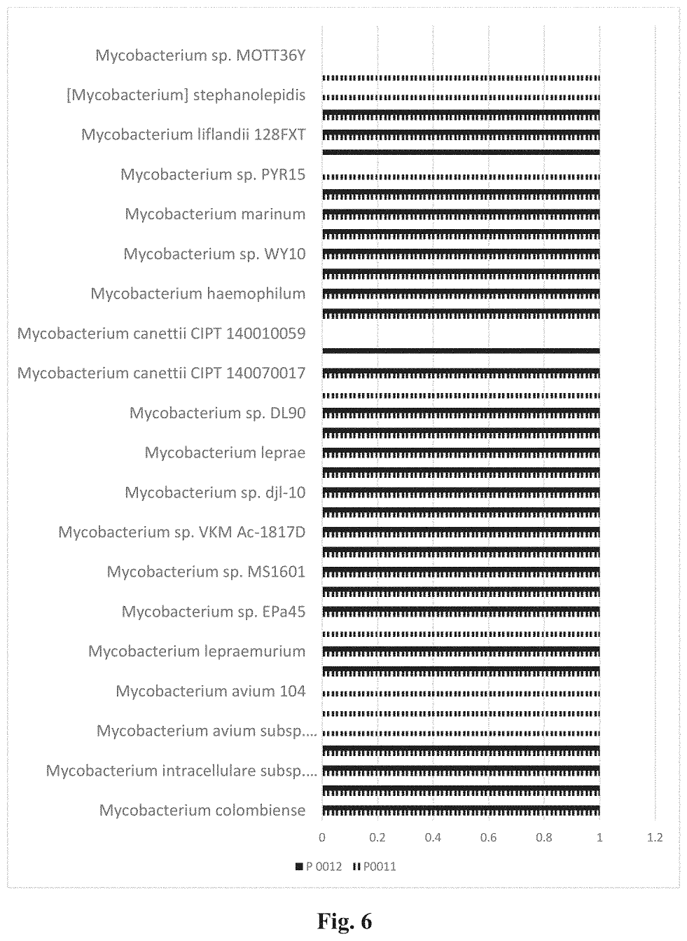

FIG. 6 is a graphical representation of the mycobacterium of patient 12 compared to patient 12's biological mother (patient 11).

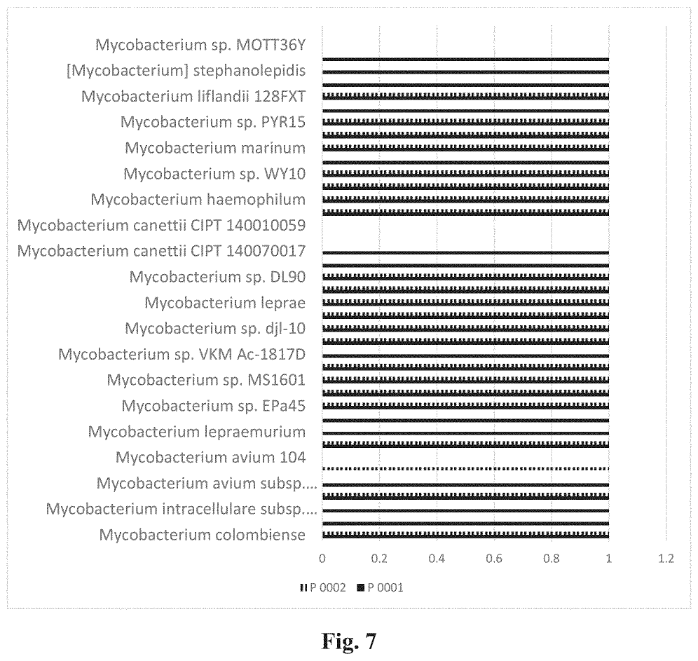

FIG. 7 is a graphical representation of mycobacterium of patient 2 compared to patient 2's biological mother (patient 1).

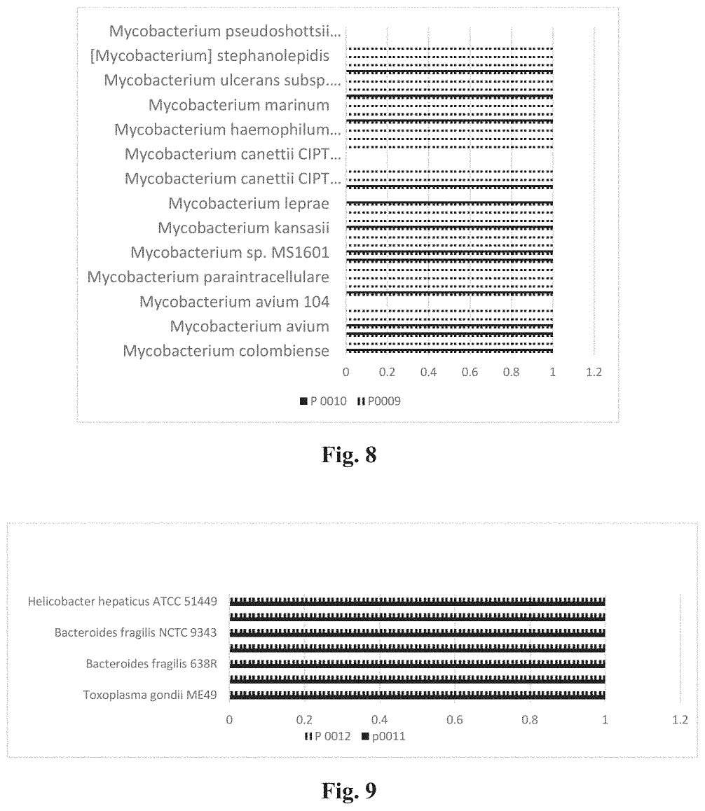

FIG. 8 is a graphical representation of the mycobacterium of patient 10 versus patient 10's biological mother (patient 9).

Table 7, shown below, documents the possible causes of Crohn's disease.

TABLE 7

| | 1 | 6 | 9 | 11 | Total |
|---|---|---|---|---|---|
| Organism Name | | | | | |
| Toxoplasma gondii ME49 | 1 | 1 | 1 | 1 | 4 |
| Bacteroides fragilis | 1 | 1 | 1 | 1 | 4 |
| Bacteroides fragilis 638R | 1 | 1 | 1 | 1 | 4 |
| Organism Name Cont. | | | | | |
| Bacteroides fragilis YCH46 | 1 | 1 | 1 | 1 | 4 |
| Bacteroides fragilis NCTC 9343 | 1 | 1 | 1 | 1 | 4 |
| Helicobacter hepaticus | 1 | 1 | 1 | 1 | 4 |
| Helicobacter hepaticus ATCC 51449 | 1 | 1 | 1 | 1 | 4 |
| | 7 | 7 | 7 | 7 | |

Table 8, shown below, documents the possible causes of Crohn's disease.

TABLE 8

| Organism Name | 2 | 3 | 5 | 10 | 12 | 13 | 14 | Total |
|---|---|---|---|---|---|---|---|---|
| Toxoplasma gondii ME49 | 1 | 1 | 1 | 1 | 1 | 1 | 1 | 7 |
| Bacteroides fragilis | 1 | 1 | 1 | 1 | 1 | 1 | 1 | 7 |
| Bacteroides fragilis 638R | 1 | 0 | 1 | 1 | 1 | 1 | 1 | 6 |
| Bacteroides fragilis YCH46 | 1 | 0 | 1 | 1 | 1 | 1 | 1 | 6 |
| Bacteroides fragilis NCTC 9343 | 1 | 0 | 1 | 1 | 1 | 1 | 1 | 6 |
| Helicobacter hepaticus | 1 | 0 | 1 | 0 | 1 | 1 | 1 | 5 |
| Helicobacter hepaticus ATCC | 1 | 0 | 1 | 0 | 1 | 1 | 1 | 5 |
| 51449 | | | | | | | | |
| | 7 | 2 | 7 | 5 | 7 | 7 | 7 | |

Table 9, shown below, documents the possible causes of Crohn's disease.

TABLE 9

| | 1 | 6 | 9 | 11 | Total |
|---|---|---|---|---|---|
| Organism Name | | | | | |
| Yersinia enterocolitica | 1 | 1 | 1 | 1 | 4 |
| Yersinia similis | 1 | 1 | 1 | 1 | 4 |
| Yersinia pseudotuberculosis | 1 | 1 | 1 | 1 | 4 |
| Yersinia pestis | 1 | 1 | 1 | 1 | 4 |
| Yersinia pestis Antiqua | 1 | 1 | 0 | 0 | 2 |
| Yersinia pestis Angola | 0 | 0 | 0 | 0 | 0 |
| Yersinia pestis str. Pestoides B | 0 | 1 | 0 | 0 | 1 |
| Yersinia pestis 3770 | 0 | 0 | 0 | 0 | 0 |
| Organism Name Cont. | | | | | |
| Yersinia pestis 2944 | 0 | 0 | 0 | 0 | 0 |
| Yersinia pestis 790 | 0 | 0 | 0 | 0 | 0 |
| Yersinia pestis 1045 | 0 | 0 | 0 | 0 | 0 |
| Yersinia pestis D182038 | 0 | 0 | 0 | 0 | 0 |
| Yersinia entomophaga | 1 | 1 | 1 | 1 | 4 |
| Yersinia ruckeri | 1 | 0 | 1 | 1 | 3 |
| Yersinia frederiksenii | 1 | 1 | 1 | 1 | 4 |
| Yersinia rohdei | 1 | 1 | 1 | 1 | 4 |
| Yersinia aldovae 670-83 | 1 | 1 | 1 | 1 | 4 |
| Yersinia aleksiciae | 1 | 1 | 1 | 1 | 4 |
| Yersinia sp. CFS1934 | 1 | 1 | 1 | 1 | 4 |
| Yersinia massiliensis | 0 | 1 | 0 | 1 | 2 |
| Yersinia kristensenii | 0 | 0 | 0 | 1 | 1 |
| Yersinia intermedia | 0 | 1 | 0 | 0 | 1 |
| | 12 | 14 | 11 | 13 | 12.5 |

Table 10, shown below, documents the possible causes of Crohn's disease.

TABLE 10

| Organism Name | 2 | 3 | 5 | 10 | 12 | 13 | 14 | Total |
|---|---|---|---|---|---|---|---|---|
| Yersinia enterocolitica | 1 | 1 | 1 | 1 | 1 | 1 | 1 | 7 |
| Yersinia similis | 1 | 1 | 1 | 1 | 1 | 1 | 1 | 7 |
| Yersinia | 1 | 1 | 1 | 1 | 1 | 1 | 1 | 7 |
| pseudotuberculosis | | | | | | | | |
| Yersinia pestis | 1 | 1 | 1 | 0 | 1 | 1 | 1 | 6 |
| Yersinia pestis Antiqua | 0 | 0 | 1 | 0 | 0 | 0 | 0 | 1 |
| Yersinia pestis Angola | 1 | 0 | 1 | 0 | 0 | 0 | 0 | 2 |
| Yersinia pestis str. | 1 | 0 | 0 | 0 | 0 | 0 | 0 | 1 |
| Pestoides B | | | | | | | | |
| Yersinia pestis 3770 | 0 | 1 | 0 | 0 | 0 | 1 | 0 | 2 |
| Yersinia pestis 2944 | 0 | 1 | 1 | 0 | 0 | 0 | 0 | 2 |
| Yersinia pestis 790 | 0 | 0 | 1 | 0 | 0 | 0 | 0 | 1 |
| Yersinia pestis 1045 | 0 | 0 | 0 | 0 | 0 | 1 | 0 | 1 |
| Yersinia pestis D182038 | 0 | 0 | 0 | 0 | 1 | 0 | 0 | 1 |
| Yersinia entomophaga | 1 | 1 | 1 | 1 | 1 | 1 | 1 | 7 |
| Yersinia ruckeri | 1 | 1 | 1 | 1 | 0 | 1 | 1 | 6 |
| Yersinia frederiksenii | 1 | 1 | 1 | 1 | 1 | 1 | 1 | 7 |
| Yersinia rohdei | 0 | 1 | 1 | 0 | 1 | 1 | 1 | 5 |
| Yersinia aldovae 670-83 | 1 | 1 | 1 | 1 | 1 | 1 | 1 | 7 |
| Yersinia aleksiciae | 0 | 0 | 1 | 0 | 1 | 1 | 1 | 4 |
| Yersinia sp. CFS1934 | 0 | 1 | 1 | 1 | 1 | 1 | 0 | 5 |
| Yersinia massiliensis | 1 | 0 | 1 | 0 | 1 | 0 | 0 | 3 |
| Yersinia kristensenii | 0 | 1 | 1 | 0 | 0 | 0 | 0 | 2 |
| Yersinia intermedia | 0 | 0 | 1 | 0 | 1 | 0 | 0 | 2 |
| | 11 | 13 | 18 | 8 | 13 | 13 | 10 | 12.2857143 |

FIG. 9 is a graphical representation of a comparison of the microbiome between patient 12 and patient 12's biological mother (patient 11).

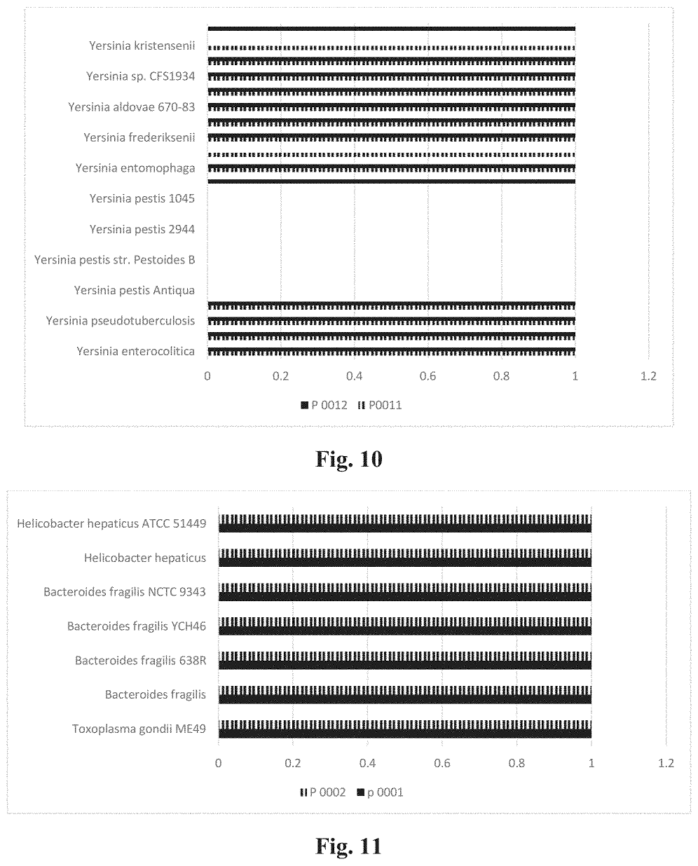

FIG. 10 shows a graphical representation of a comparison of the microbiome between patient 12 and patient 12's biological mother (patient 11).

FIG. 11 shows a graphical representation of a comparison of the microbiome between patient 2 and patient 2's biological mother (patient 1).

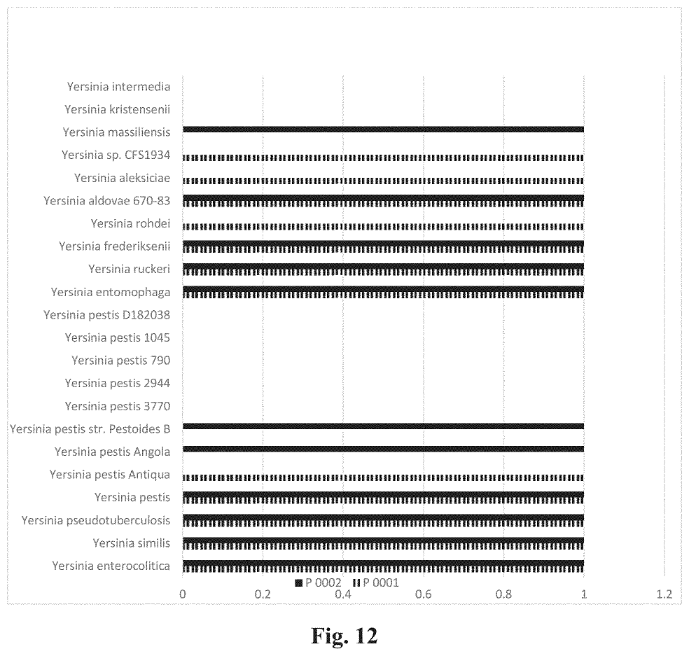

FIG. 12 shows a graphical representation of a comparison of the microbiome between patient 2 and patient 2's biological mother (patient 1).

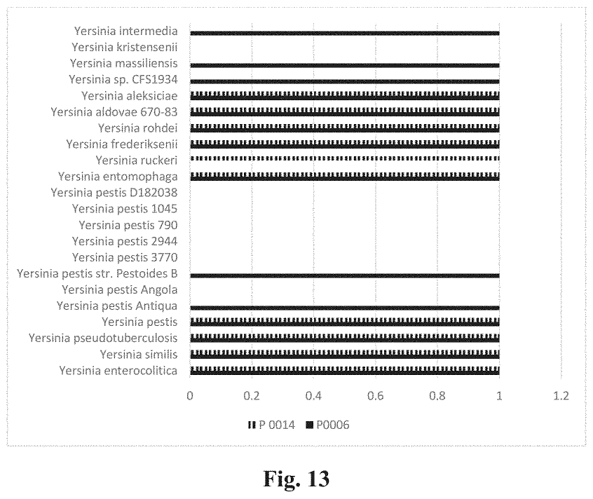

FIG. 13 shows a graphical representation of a comparison of the microbiome between patient 14 and patient 14's biological brother (patient 6).

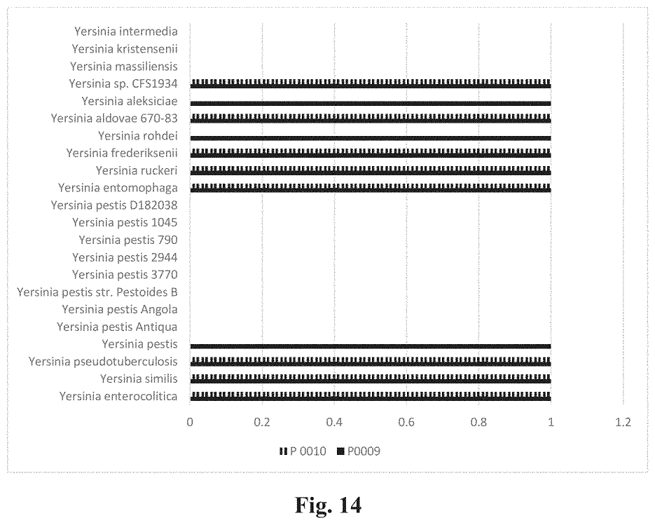

FIG. 14 shows a graphical representation of a comparison of the microbiome between patient 10 and patient 10's biological mother (patient 9).

Table 11, shown below, documents common organisms found in patients with Crohn's disease.

TABLE 11

| FAMILIES | | | |
|---|---|---|---|
| FT-0001-500ng | s_Toxoplasma gondii | 59 | FT-0001 |
| FT-0002 | s_Toxoplasma gondii | 36,278 | FT-0002 |
| FT-0006 | s_Toxoplasma gondii | 68 | FT-0006 |
| FT-0014 | s_Toxoplasma gondii | 14,312 | FT-0014 |
| FT-0009 | s_Toxoplasma gondii | 32 | FT-0009 |
| FT-0010 | s_Toxoplasma gondii | 31,855 | FT-0010 |
| FT-0011 | s_Toxoplasma gondii | 52 | FT-0011 |
| FT-0012 | s_Toxoplasma gondii | 1,425 | FT-0012 |

Table 12, shown below, documents common organisms found in patients with Crohn's disease.

TABLE 12

| CHRON | | |
|---|---|---|
| FT-0002 | s_Toxoplasma gondii | 36,278 |
| FT-0003 | s_Toxoplasma gondii | 19,625 |
| FT-0005 | s_Toxoplasma gondii | 206 |
| FT-0010 | s_Toxoplasma gondii | 31,855 |
| FT-0012 | s_Toxoplasma gondii | 1,425 |
| FT-0013 | s_Toxoplasma gondii | 22,864 |
| FT-0014 | s_Toxoplasma gondii | 14,312 |

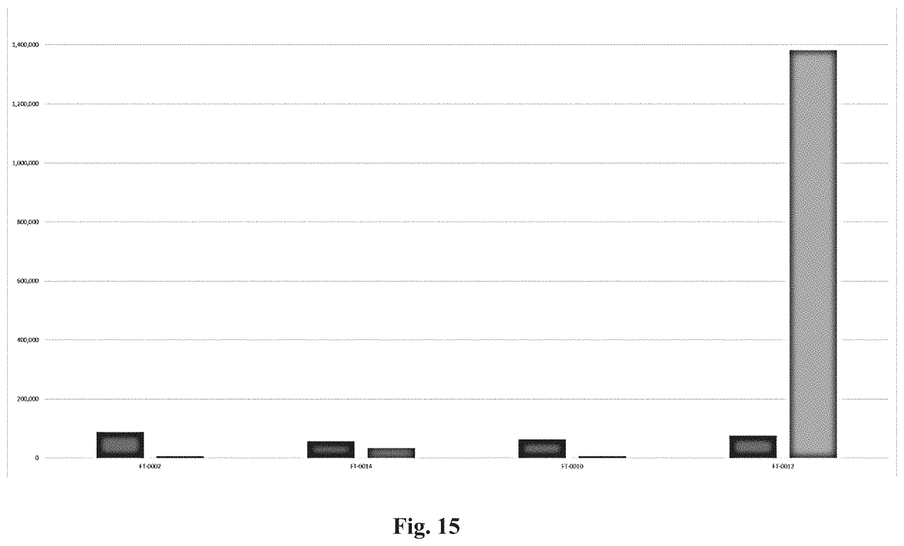

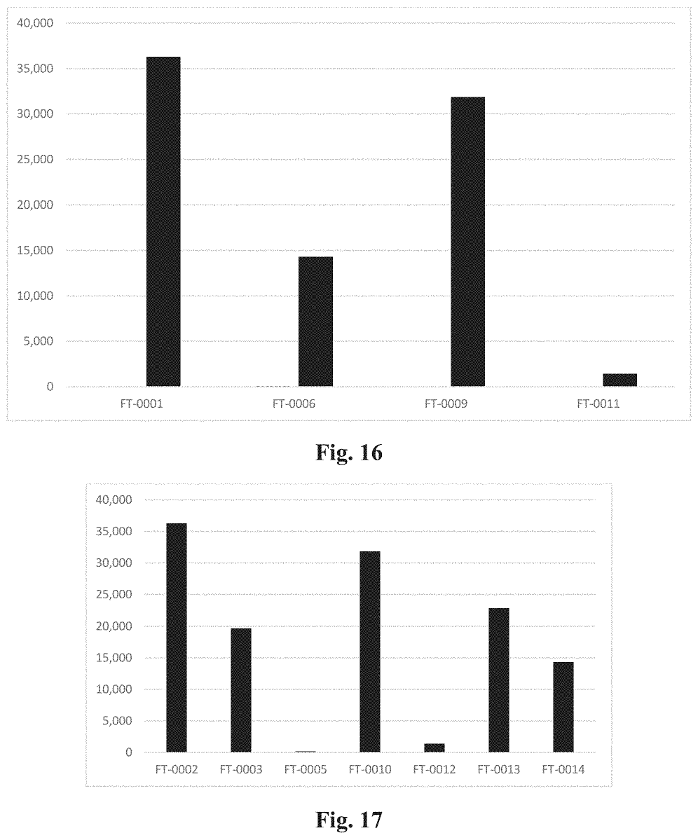

FIGS. 15, 16 and 17 are graphical representations of common microbes found in patients with Crohn's disease. More specifically, FIG. 15 shows the amount of Bacteroides fragilis found in patients with Crohn's disease as compared to healthy family members, and FIGS. 16 and 17 show the amounts of Toxoplasma gondii found in patients with Crohn's disease.

Table 13, shown below, documents common organisms found in patients with Crohn's disease.

TABLE 13

| FAMILIES | | | |
|---|---|---|---|
| FT-0001-500ng | s_Escherichia coli | 603 | FT-0001 |
| FT-0002 | s_Escherichia coli | 239,346 | FT-0002 |
| FT-0006 | s_Escherichia coli | 121,584 | FT-0006 |
| FT-0014 | s_Escherichia coli | 6,501 | FT-0014 |
| FT-0009 | s_Escherichia coli | 486 | FT-0009 |
| FT-0010 | s_Escherichia coli | 174,401 | FT-0010 |
| FT-0011 | s_Escherichia coli | 405 | FT-0011 |
| FT-0012 | s_Escherichia coli | 1,589 | FT-0012 |

Table 14, shown below, documents common organisms found in patients with Crohn's disease.

TABLE 14

| CHRON | | |
|---|---|---|
| FT-0002 | s_Escherichia coli | 239,346 |
| FT-0003 | s_Escherichia coli | 31,164 |
| FT-0005 | s_Escherichia coli | 330,582 |
| FT-0010 | s_Escherichia coli | 174,401 |
| CHRON Cont. | | |
| FT-0012 | s_Escherichia coli | 1,589 |
| FT-0013 | s_Escherichia coli | 91,329 |
| FT-0014 | s_Escherichia coli | 6,501 |

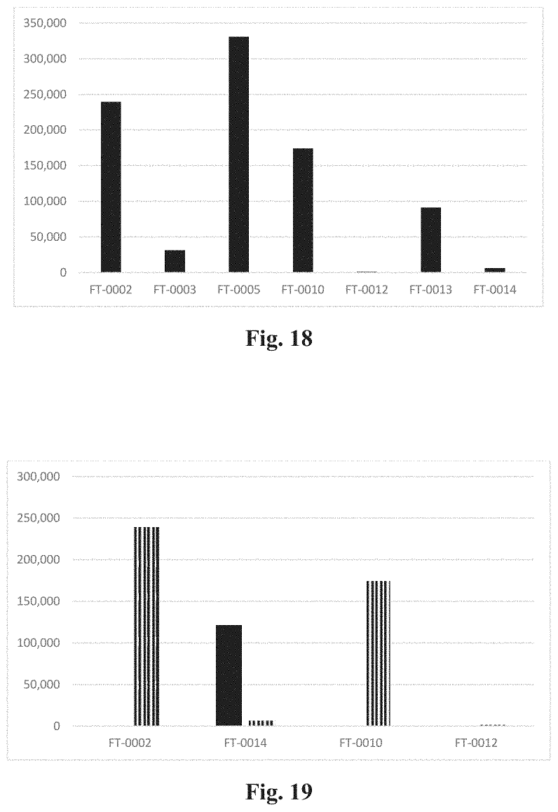

FIG. 18 is a graphical representation showing the amount of Escherichia coli found in patients with Crohn's disease.

FIG. 19 is a graphical representation showing the amount of Escherichia coli found in patients with Crohn's disease (shown with the solid black bars) and healthy family members of those patients (shown with the series of solid black bars).

Table 15, shown below, documents common organisms found in patients with Crohn's disease.

TABLE 15

| FAMILIES | | | |
|---|---|---|---|
| FT-0001-500ng | s_Bacteroides fragilis | 86,801 | FT-0001 |
| FT-0002 | s_Bacteroides fragilis | 6,461 | FT-0002 |
| FT-0006 | s_Bacteroides fragilis | 56,124 | FT-0006 |
| FT-0014 | s_Bacteroides fragilis | 33,504 | FT-0014 |
| FT-0009 | s_Bacteroides fragilis | 63,219 | FT-0009 |
| FT-0010 | s_Bacteroides fragilis | 4,636 | FT-0010 |
| FT-0011 | s_Bacteroides fragilis | 75,387 | FT-0011 |
| FT-0012 | s_Bacteroides fragilis | 1,382,505 | FT-0012 |

Table 16, shown below, documents common organisms found in patients with Crohn's disease.

TABLE 16

| CHRON | | |
|---|---|---|
| FT-0002 | s_Bacteroides fragilis | 6,461 |
| FT-0003-500ng | s_Bacteroides fragilis | 4 |
| FT-0005 | s_Bacteroides fragilis | 54,107 |
| FT-0010 | s_Bacteroides fragilis | 4,636 |
| FT-0012 | s_Bacteroides fragilis | 1,382,505 |
| FT-0013 | s_Bacteroides fragilis | 31,886 |
| FT-0014 | s_Bacteroides fragilis | 33,504 |

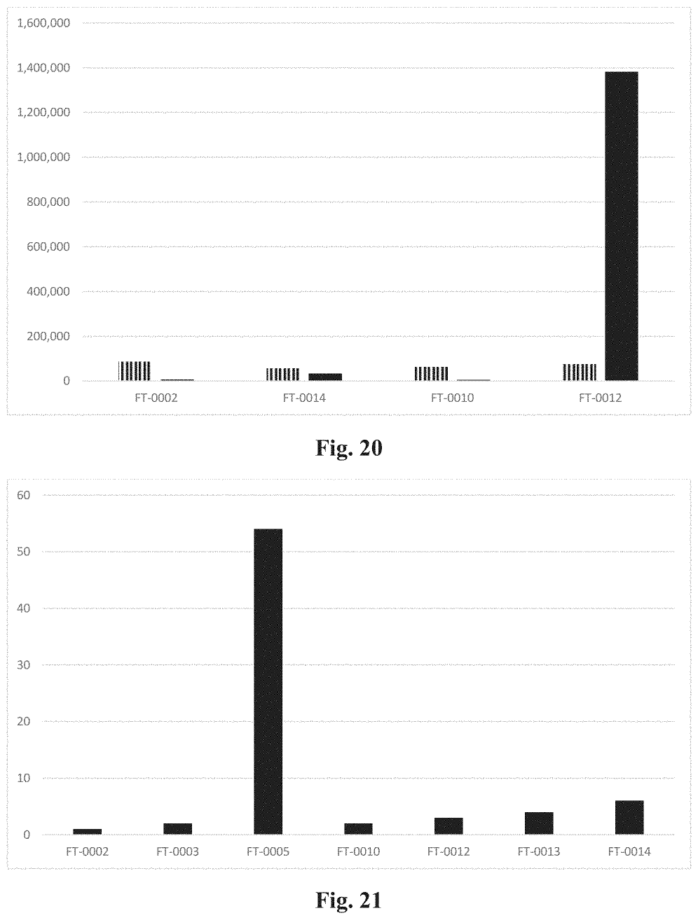

FIG. 20 is a graphical representation showing the amount of Bacteroides fragilis found in patients with Crohn's disease (shown in solid back bars) as compared to healthy family members (shown via a series of black bars).

Table 17, shown below, documents common organisms found in patients with Crohn's disease.

TABLE 17

| FAMILIES | | |
|---|---|---|
| FT-0001-500ng | s_Mycobacterium avium | 40 |
| FT-0002 | s_Mycobacterium avium | 1 |
| FT-0006 | s_Mycobacterium avium | 35 |
| FT-0014 | s_Mycobacterium avium | 6 |
| FT-0009 | s_Mycobacterium avium | 28 |
| FT-0010 | s_Mycobacterium avium | 2 |
| FT-0011 | s_Mycobacterium avium | 56 |
| FT-0012 | s_Mycobacterium avium | 3 |

Table 18, shown below, documents common organisms found in patients with Crohn's disease.

TABLE 18

| CHRON | | | |
|---|---|---|---|
| FT-0002 | s_Mycobacterium avium | 1 | FT-0002 |
| FT-0003-500ng | s_Mycobacterium avium | 2 | FT-0003 |
| FT-0005 | s_Mycobacterium avium | 54 | FT-0005 |
| FT-0010 | s_Mycobacterium avium | 2 | FT-0010 |
| FT-0012 | s_Mycobacterium avium | 3 | FT-0012 |
| FT-0013 | s_Mycobacterium avium | 4 | FT-0013 |
| FT-0014 | s_Mycobacterium avium | 6 | FT-0014 |

FIG. 21 is a graphical representation showing common organisms found in patients with Crohn's disease. More specifically, FIG. 21 shows Crohn's Patients with Mycobacterium avium subspecies paratuberculosis

Table 19, shown below, documents common organisms found in patients with Crohn's disease.

TABLE 19

| FAMILIES | | |
|---|---|---|
| FT-0001-500ng | s_Helicobacter hepaticus | 36 |
| FT-0002 | s_Helicobacter hepaticus | 1 |
| FT-0006 | s_Helicobacter hepaticus | 18 |
| FAMILIES Cont. | | |
| FT-0014 | s_Helicobacter hepaticus | 5 |
| FT-0009 | s_Helicobacter hepaticus | 69 |
| FT-0010 | s_Helicobacter hepaticus | 0 |
| FT-0011 | s_Helicobacter hepaticus | 2 |
| FT-0012 | s_Helicobacter hepaticus | 5 |

Table 20, shown below, documents common organisms found in patients with Crohn's disease.

TABLE 20

| CHRON | | |
|---|---|---|
| FT-0002 | s_Helicobacter hepaticus | 1 |
| FT-0003-500ng | s_Helicobacter hepaticus | 0 |
| FT-0005 | s_Helicobacter hepaticus | 5 |
| FT-0010 | s_Helicobacter hepaticus | 0 |
| FT-0012 | s_Helicobacter hepaticus | 5 |
| FT-0013 | s_Helicobacter hepaticus | 9 |
| FT-0014 | s_Helicobacter hepaticus | 5 |

Table 21, shown below, documents common organisms found in patients with Crohn's disease.

TABLE 21

| FAMILIES | | |
|---|---|---|
| FT-0001-500ng | s_Enterococcus faecalis | 36 |
| FT-0002 | s_Enterococcus faecalis | 445 |
| FT-0006 | s_Enterococcus faecalis | 150 |
| FT-0014 | s_Enterococcus faecalis | 31 |
| FT-0009 | s_Enterococcus faecalis | 150 |
| FT-0010 | s_Enterococcus faecalis | 20 |
| FT-0011 | s_Enterococcus faecalis | 247 |
| FT-0012 | s_Enterococcus faecalis | 193 |

Table 22, shown below, documents common organisms found in patients with Crohn's disease.

TABLE 22

| CHRON | | |
|---|---|---|
| FT-0002 | s_Enterococcus faecalis | 36 |
| FT-0003-500ng | s_Enterococcus faecalis | 0 |
| FT-0005 | s_Enterococcus faecalis | 501 |
| FT-0010 | s_Enterococcus faecalis | 20 |
| FT-0012 | s_Enterococcus faecalis | 193 |
| FT-0013 | s_Enterococcus faecalis | 58 |
| FT-0014 | s_Enterococcus faecalis | 31 |

Table 23, shown below, documents common organisms found in patients with Crohn's disease.

TABLE 23

| | 1 | 2 | 11 | 12 | 14 | 6 |
|---|---|---|---|---|---|---|
| Patient ID | | | | | | |
| Enterococcus faecalis V583 | 1 | 1 | 1 | 1 | 1 | 1 |
| Enterococcus faecalis D32 | 0 | 0 | 1 | 1 | 1 | 1 |
| Enterococcus faecalis ARO1/DG | 1 | 0 | 1 | 0 | 0 | 1 |
| Enterococcus faecalis DENG1 | 0 | 0 | 1 | 1 | 0 | 0 |
| Enterococcus faecalis ATCC | 1 | 0 | 1 | 0 | 1 | 1 |
| Patient ID Cont. | | | | | | |
| Enterococcus faecalis str. | 1 | 0 | 1 | 1 | 0 | 0 |
| Symbioflor 1 | | | | | | |
| Mycobacterium avium subsp. | 1 | 0 | 1 | 1 | 0 | 0 |
| paratuberculosis | | | | | | |
| Malassezia furfur | 0 | 0 | 0 | 0 | 0 | 0 |

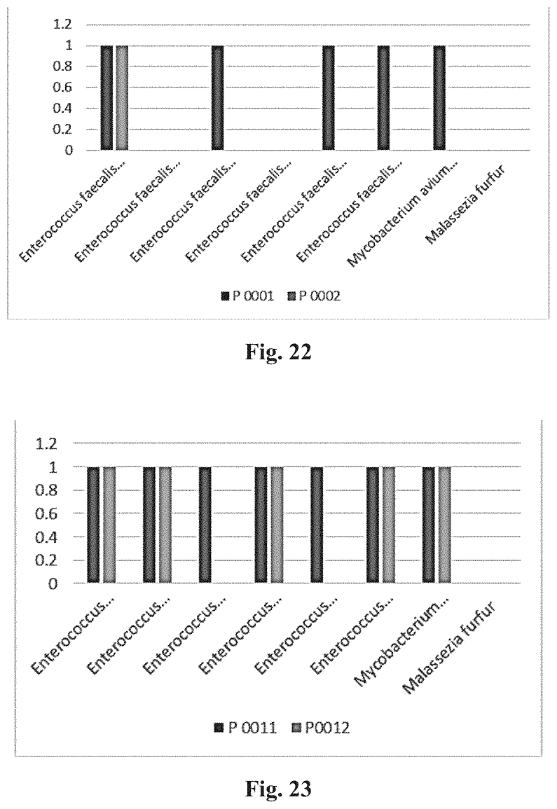

FIG. 22 is a graphical representation of a comparison of the microbiome between patient 1 and patient 1's biological mother (patient 2).

FIG. 23 is a graphical representation of a comparison of the microbiome between patient 12 and patient 12's biological mother (patient 11).

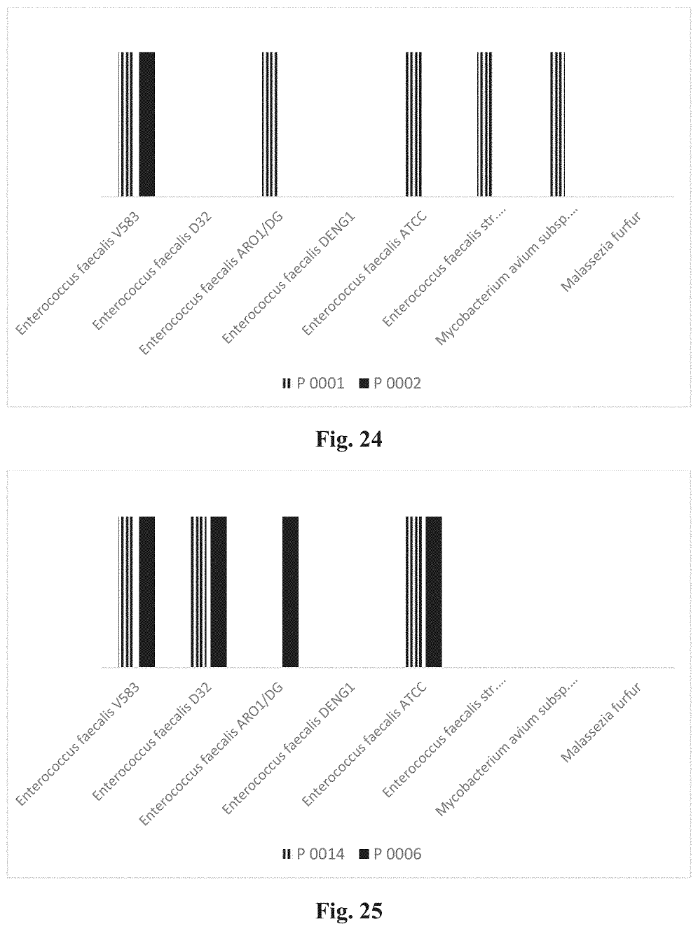

FIG. 24 is a graphical representation of a comparison of the microbiome between patient 2 and patient 2's biological mother (patient 1).

FIG. 25 is a graphical representation of a comparison of the microbiome between patient 14 and patient 14's biological brother (patient 6).

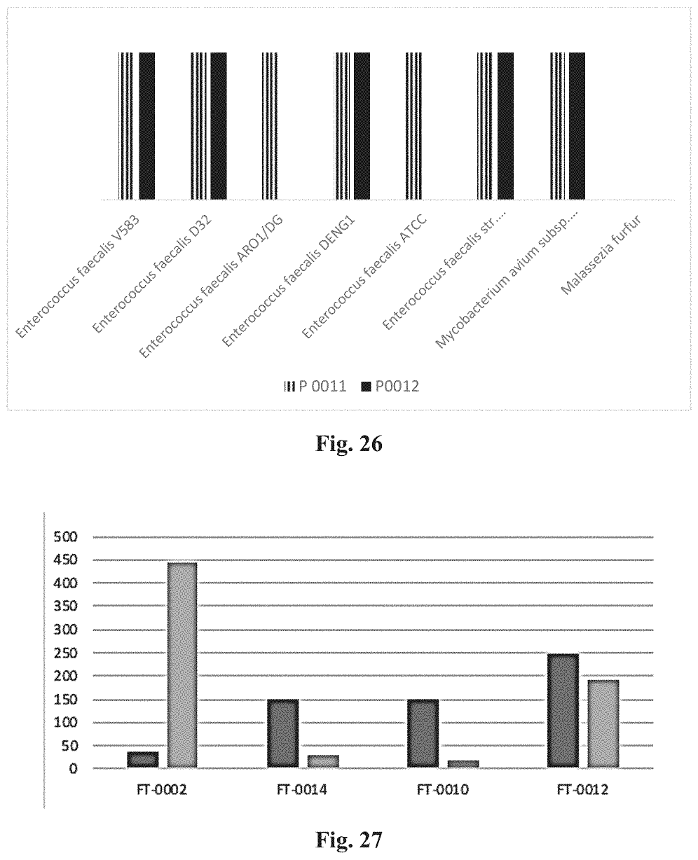

FIG. 26 is a graphical representation of a comparison of the microbiome between patient 12 and patient 12's biological mother (patient 11).

FIG. 27-29 are graphical representations showing common organisms found in patients with Crohn's disease. More specifically, FIG. 27 shows a comparison of the amount of Enterococcus faecalis found in patients with Crohn's disease (shown via the darker bars) and healthy family members (shown via the lighter bars).

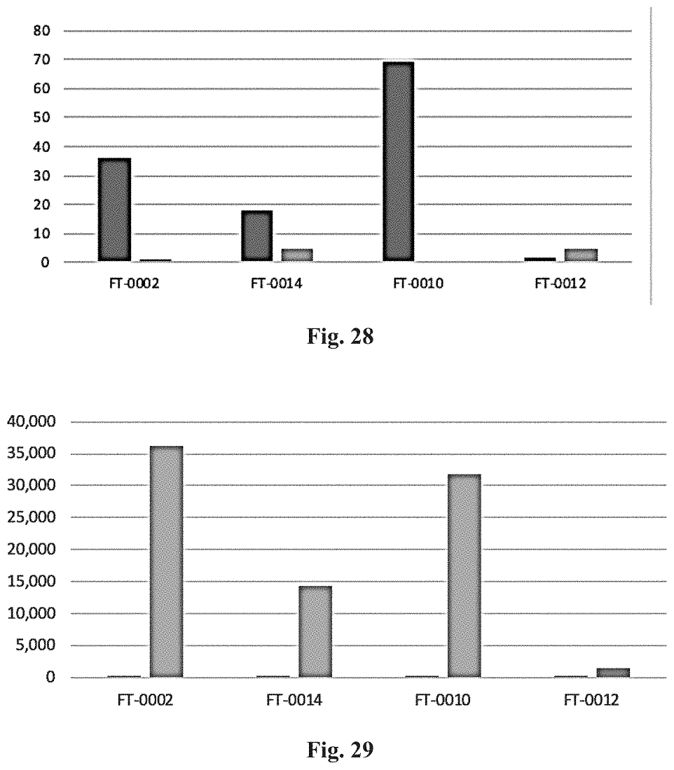

FIG. 28 shows a comparison of the amount of Helicobacter heptaticus found in patients with Crohn's disease (shown via the lighter bars) and healthy family members (shown via the darker bars).

FIG. 29 shows a comparison of the amount of Toxoplasma gondii found in patients with Crohn's disease (shown via the lighter bars) and healthy family members (shown via the darker bars, but the darker bars are near zero and are difficult to view).

### Example 2: Chronic Urinary Tract Infection

Chronic urinary tract infections (UTIs) are painful and frustrating for patients. The symptoms of a lower urinary tract include frequent and/or urgent need to urinate, dysuria, soreness in the lower abdomen, back, or sides, pain on urination, need to urinate at night, and urine that is discolored potentially with a foul odor. If the infection is in the kidneys it can be life threatening. There are many proposed causes of chronic UTIs, however some studies have indicated that dysbiosis of the gut microbiome may play a role. The objective of this example is to analyze the microbiome of patients with chronic UTIs to look for similarities in relative abundance of microbes and groups of microbes.

The same procedure noted above for Example 1 was performed on 30 individuals suffering from chronic urinary tract infection.

### Example 3: Clostridoides difficile Infection

Clostridoides difficile is a gram-positive spore-forming rod-shaped bacterium which can cause severe illness. Infection with C. difficile frequently occurs following antibiotic use, suggesting that dysbiosis, or an imbalance of the microbiome of the gut, could play a major role in the development of infection. The objective of this example is to correlate conditions in the microbiome which could contribute to, or be the result of, infection with C. difficile.

The same procedure noted above for Example 1 was performed on 30 individuals suffering from Clostridoides difficile infection. The following are criteria for moderate to severe Clostridoides difficile infection:
1. Leukocytosis (white blood cell count >20×109/L)
2. Plasma albumin level <30 g/L
3. Creatinine level >50% of baseline
4. Hypotension (systolic blood pressure <100 mmHg)
5. Fever (temperature >38° C.)
6. Abdominal pain and distension
7. Radiological evidence of colonic dilation, ascites or ileus

### Example 4: Obesity

Obesity is associated with myriad sequelae including type II diabetes, cardiovascular disease, some cancers, kidney disease, obstructive sleep apnea, gout, osteoarthritis, and many others. These frequently lead to a shortened lifespan. There is a strong positive correlation between weight loss and reduction of risk for these conditions. Studies of fecal microbiota transplantation have shown that the procedure has the ability instigate obesity. This suggests that there is a microbiome component to obesity. Obesity is defined as a Body Mass Index (BMI) of >30 kg/m³. The objective of this example is to investigate the microbiome of obese individuals to examine the relative abundance of microbes contained therein.

The same procedure noted above for Example 1 was performed on 30 individuals suffering from obesity.

### Example 5: Alzheimer's Disease

Alzheimer's disease (AD) is a neurodegenerative disorder and is the most common form of dementia. As of 2014 there were more than 5 million Americans living with Alzheimer's disease. The characteristic brain lesions, amyloid plaques and neurofibrillary tangles, cause progressive loss of cognitive function. The gut may play a major roll in this process. Dysbiosis of the gut microbiome can lead to systemic inflammation, which may in turn compromise the blood brain barrier, and lead to neuroinflammation and damage to neurons. The objective of this example to determine whether a specific microbe is present in individuals with Alzheimer's disease.

The same procedure noted above for Example 1 was performed on individuals suffering from Alzheimer's disease.

### Example 6: Psoriasis

Psoriasis is a long-term skin autoimmune disease which causes patches of red, itchy, scaly skin. These patches can be small and localized or widespread. Plaque Psoriasis is the most common type, accounting for 90% of cases. The most commonly affected areas are the forearms, skins, naval area, and scalp. While it is thought that genetics may play a role in the development of Psoriasis, early sequencing studies of the gut microbiome of Psoriasis patients have found the relative abundance of certain microbes to be altered in Psoriasis patients. Thus, the balance of the microbiome may play an important role in Psoriasis development and treatment. The objective of this example to evaluate the similarities in the gut flora of different individuals with psoriasis and difference when compared to healthy individuals.

The same procedure noted above for Example 1 was performed on 30 individuals suffering from psoriasis.

### Example 7: Autism

Autism spectrum disorders (ASD) are characterized by qualitative impairment in social interaction and communication skills, as well as stereotypic behaviors and limited activities and interests. As of 2014, 1 in 59 children in the United States will be diagnosed with ASD. In one sample set taken from several locations in the US, the rate of ASD diagnosis went from 1 in 150 to 1 in 68 in just 10 years, more than doubling. Core features of ASDs include verbal and nonverbal communication impairments, qualitative impairments in social interaction and the presence of maladaptive routines, repetitive behaviors and atypical interests or fixations. Comorbidity with at least one gastrointestinal symptom occurs in almost half of all children with ASD. The degree of severity of gastrointestinal symptoms strongly correlates to the degree of autism symptom severity. While some studies have identified specific microbes or families of microbes found to be perturbed in patients with ASD, evidence supporting positive impacts of altering the microbiome of individuals with ASD is in the very early stages. In one small study of oral vancomycin, short term improvement was seen with the majority of subjects, hinting at the strength of the gut-brain axis in the severity of ASD symptoms. The objective of this example is to evaluate the similarities in the gut flora of different individuals with autism and differences when compared to healthy individuals.

The same procedure noted above for Example 1 was performed on 30 individuals suffering from autism.

### Example 8: Myalgic Encephalomyelitis/Chronic Fatigue Syndrome

Chronic Fatigue Syndrome (CFS), also known as Myalgic Encephalomyelitis(ME) or ME/CFS, is a debilitating illness with no known cause, and no true treatment options. It also has no known cure. Patients with ME/CFS experience profound exhaustion, unrefreshing sleep, joint aches and pains, post-exertional malaise, and frequently gastrointestinal problems. In a survey of drug use by ME/CFS patients there was found to be greater use of antacids, H2 blockers, and proton pump inhibitors than in the general population. Bacteriotherapy using oral and rectal probiotics has caused some improvement in patient's gastrointestinal symptoms. Thus dysbiosis is hypothesized to play a role in ME/CFS. The objective of this example is to evaluate the similarities in the gut flora of different individuals with ME/CFS and differences when compared to healthy individuals.

The same procedure noted above for Example 1 was performed on 30 individuals suffering from ME/CFS.

### Example 9: The Role of Diet

The human gastrointestinal (GI) microbiome is a complex, interconnected web of microbes, living in a symbiotic relationship with their host. There are greater than ten times more bacteria in the human body than there are human cells, all in a delicate and ever-changing balance to maintain a healthy GI tract. When this balance is disrupted, a condition known as dysbiosis, disease can occur. There is still a debate over whether dysbiosis is a cause of disease or a symptom of it. Naturally, since the microbiome has such a profound impact on human health, including helping humans digest food, make vitamins, and teach their immune cells to recognize pathogens, there is a desire to study and learn as much about the microbiome as possible. By correlating this data with survey data and medical records for the patients, connections may begin to be drawn between organisms present in the microbiome of the gastrointestinal tract, and disease. This is accomplished by comparing the answers of survey questions to disease states in participants. For example, if there is one particular microbe in patients with Crohn's disease, the data suggest that this microbe could play a role in the cause or progression of this disease. More importantly, only microbial activity within a family can be compared. The microbiome is passed on from mother to child therefore it makes sense to compare microbiome of mother and child to understand better the microbiome. Much like fingerprints, no microbiome is identical therefore, in order to understand a disease, it is preferred to look at the microbiome of a parent compared to a child or in an individual at baseline of healthy compared to a disease state. The objective of this example is to evaluate the similarities in the gut flora of different individuals with similar diet.

The same procedure noted above for Example 1 was performed on 30 individuals with similar diet.

### Example 10: COVID-19 Infection

COVID-19 is caused by a novel betacoronavirus (SARS-CoV-2) that is thought to have originated in bats in the city of Wuhan, China. This disease has rapidly spread to become a worldwide pandemic. Scientists have identified the molecular structure of the spike glycoproteins on the surface of the virus, which are what allow the virus to “stick” to its target, in this case the human lung. The virus has a very similar sequence and structure to the SARS coronaviruses, with the exception of the receptor binding domain. Within a specific loop domain of the binding pocket of SARS-CoV-2, there is a change which replaces two proline residues with two flexible glycine residues, converting a rigid structure to something much more flexible, which is thought to facilitate stronger binding to the human host cell ACE2 receptor. The ACE2 receptor is present in the lungs, however, it is also present in the intestine, kidneys, and testis. Thus, there is concern that the intestines could be a reservoir for the virus, and that the virus could be transmitted by the fecal oral route, in addition to transmission by aerosols. It is critically important that patient stools be tested to determine if this is happening.

There are many diseases for which the degree of dysbiosis is a marker for disease severity. It is highly likely this phenomenon will also exist in the case of COVID-19. Thus, comparison between patients with different levels of severity will allow determination of whether it occurs with COVID-19. The objective of this example to determine whether the virus is shed in the stool following negative RT-PCR testing and to correlate the microbiome sequencing data with information provided by patients and their medical records regarding COVID-19.

The procedure for this example is as follows. The first step was collection of a COVID-19 sample. Nasopharyngeal (NP) and oropharyngeal (OP) swabs were collected according to CDC protocol. Synthetic fiber swabs with plastic shafts were used. NP swabs were collected by insertion of a swab into the patient's nostril parallel to the palate. The swab is left in place a few seconds to allow it to absorb secretions. OP swabs were collected by inserting the swab into the mouth without touching the tongue, cheek, or uvula. The tip of the swab was touched to the area around the tonsils and twisted five times to collect sufficient secretions for testing.

Following a positive test by RT-PCR, and again following subsequent negative test, patient stool samples were collected via the procedures noted above (stool sample collection kit or colonoscopy). Following fecal collection, individual patient DNA and RNA was extracted and purified. The isolated DNA was quantitated utilizing a fluorometer, and the RNA was quantitated with a RNA quantitation system.

After DNA quantification, the DNA was normalized and libraries were prepared utilizing shotgun methodology. This process utilized the shotgun workflow wherein samples undergo tagmentation, amplification and indexing, and purification.

After RNA quantification, the RNA was normalized and library fabrication was executed. This workflow included RNA fragmentation, first and second strand cDNA synthesis, adenylation, adapter ligation, and amplification.

Samples libraries were normalized to create a library pool which is quantified and appropriately diluted to the final loading concentration to be sequenced on the appropriate sequencing system/machine.

Following completion of the NextSeq run, the raw.bcl data was streamed in real time for conversion to FASTQ files. The FASTQ files were then pushed through the bioinformatics metagenomics pipeline with patient specific endpoint readouts profiling each individual's unique microbiome.

More specifically, the bioinformatics pipeline utilized computational tools that profiled the microbial communities from metagenomic sequencing data with species level resolution. Patient microbiome profiles were analyzed to ascertain not only the profile of microbes in patient samples but also to identify specific strains, and provide accurate estimation of organismal abundance relative to the overall diversity.

Patient specific microbiome profiles were aligned to their medical records and other patient provided information for further analysis and interpretation.

The stool samples were retained for future use in a 20° C. freezer.

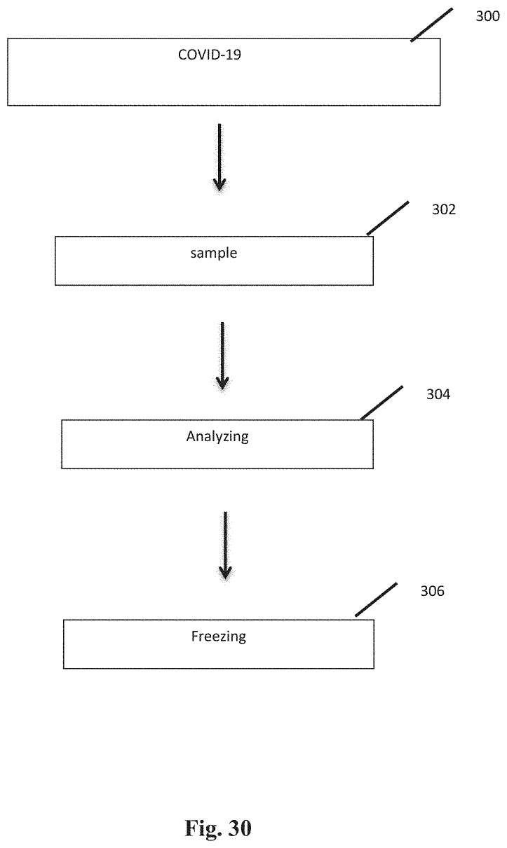

FIG. 30 is a flow chart of the method of sequencing the microbiome of an individual recovering from COVID-19 infection. The method comprises the basic steps of providing an individual that had been infected with COVID-19 300; providing a stool sample from the individual 302; analyzing the microbiome of the individual 304; and freezing the stool sample from the individual for future use 306.

### Example 11: Role of Gut Flora in Disease

The objective of this example is to investigate the microbiome of individuals suffering from the following diseases or health conditions: C. difficile infection, Obesity, Autism, Alzheimer's disease, Crohn's disease, Myalgic Encephalomyelitis/Chronic, Fatigue Syndrome (ME/CFS), Psoriasis, Chronic UTI, Ulcerative Colitis, Multiple Sclerosis (MS), Chronic constipation, Celiac sprue, Lyme disease, Elevated cholesterol, Colorectal cancer, Amyotrophic lateral sclerosis (ALS), Rheumatoid arthritis, Parkinson's disease, Depression, Anxiety, Obsessive-Compulsive disorder, Bipolar Disorder, Migraine headaches, Diabetes mellitus, Lupus, Epidermolysis, Metastatic mesothelioma, irritable bowl syndrome (IBS) Diarrhea, IBS Constipation, Eczema, Acne, Fatty liver, Myasthenia gravis, Gout.

The same procedure noted above for Example 1 was performed on at least 100 individuals suffering from each disease or health condition listed above.

### Example 12: SARS-CoV-2

Objective: SARS-CoV-2 has been detected not only in respiratory secretions, but also in stool collections. The objective of this example is to identify SARS-CoV-2 by enrichment NGS from fecal samples, and to utilize whole genome analysis to characterize SARS-CoV-2 mutational variations in COVID-19 patients.

Methods: 14 study participants (n=14) underwent testing for SARS-CoV-2 from fecal samples by whole genome enrichment NGS. Following fecal collection, RNA was extracted, reverse transcribed, and the library was prepped, enriched, and sequenced. Sequences were then mapped to the SARS-CoV-2 Wuhan-Hu-1 (MN90847.3) complete genome utilizing One Codex's SARS-CoV-2 bioinformatics analysis pipeline. SARS-CoV-2 positive samples were further analyzed for mutational variants that differed from the reference genome. Of the 14 study participants, 12 also had their nasopharyngeal swabs tested for SARS-CoV-2 by RT-PCR.

Results: Study participants underwent testing for SARS-CoV-2 from fecal samples by whole genome enrichment NGS (n=14), and RT-PCR nasopharyngeal swab analysis (n=12). The concordance of SARS-CoV-2 detection by enrichment NGS from stools with RT-PCR nasopharyngeal analysis was 100%. Unique variants were identified in four patients, with a total of 33 different mutations among those in which SARS-CoV-2 was detected by whole genome enrichment NGS.

More specifically, the results from patients that had their stool samples tested by whole genome enrichment NGS were evaluated, as well as their nasopharyngeal swabs were tested by RT-PCR for the presence of SARS-CoV-2. Of the 14 study participants, ten were symptomatic and tested positive for SARS-CoV-2 by RT-PCR, two asymptomatic individuals tested negative, and two other asymptomatic individuals did not undergo RT-PCR testing (Table 24). Patients 5 and 7, that had tested positive by RT-PCR from nasopharyngeal swabs, were treated with Hydroxychloroquine (HCQ), Azithromycin, vitamin C, vitamin D, and zinc for 10 days prior to fecal collection. Similarly, after positive nasopharyngeal swab, patient 13 was treated with vitamin C, vitamin D, and zinc for 10 days before fecal collection. The concordance of SARS-CoV-2 detection by enrichment NGS from stools among positive non-treated patients tested by RT-PCR nasopharyngeal analysis was 100% (7/7). Patient 8, who did not undergo nasopharyngeal analysis, tested positive for SARS-CoV-2 by NGS. The three patients (5, 7, 13) that received treatment prior to providing fecal samples, all tested negative by NGS. Asymptomatic patients 2 and 9, who tested negative by nasopharyngeal swab, were also negative by NGS, as was asymptomatic patient 14.

Table 24 documents the symptoms and SARS-CoV-2 testing results.

TABLE 24

| | | Nasopha- | | | |
|---|---|---|---|---|---|
| | | ryngeal | | | |
| | | Swab | | Fecal | Patient |
| Sample ID | Symptoms | (RT-PCR) | Treated | (NGS) | Location |
| Patient 1 | febrile, diarrhea, | + | no | + | PA |
| | anosmia, O2 | | | | |
| | sat. <90% | | | | |
| Patient 3 | febrile, diarrhea, | + | no | + | CA |
| | O2 sat. <90% | | | | |
| Patient 4 | febrile, diarrhea, | + | no | + | AZ |
| | anosmia, O2 | | | | |
| | sat. <90% | | | | |
| Patient 6 | febrile, cough, | + | no | + | AZ |
| | anosmia | | | | |
| Patient 8 | none | n/a | no | + | CA |
| Patient 10 | febrile, cough, | + | no | + | GA |
| | headache | | | | |
| Patient 11 | febrile, cough, | + | no | + | GA |
| | headache | | | | |
| Patient 12 | febrile, cough | + | no | + | GA |
| Patient 5 | febrile, cough | + | yes | − | CA |
| Patient 7 | febrile, cough | + | yes | − | GA |
| Patient 13 | febrile, cough | + | yes | − | GA |
| Patient 2 | none | − | no | − | CA |
| Patient 9 | none | − | no | − | CA |
| Patient 14 | none | n/a | no | − | CA |

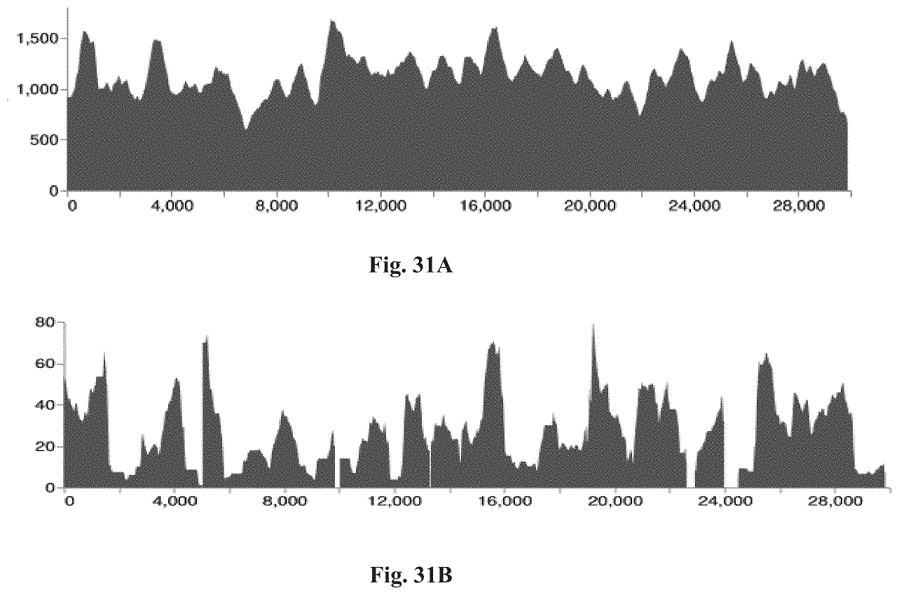

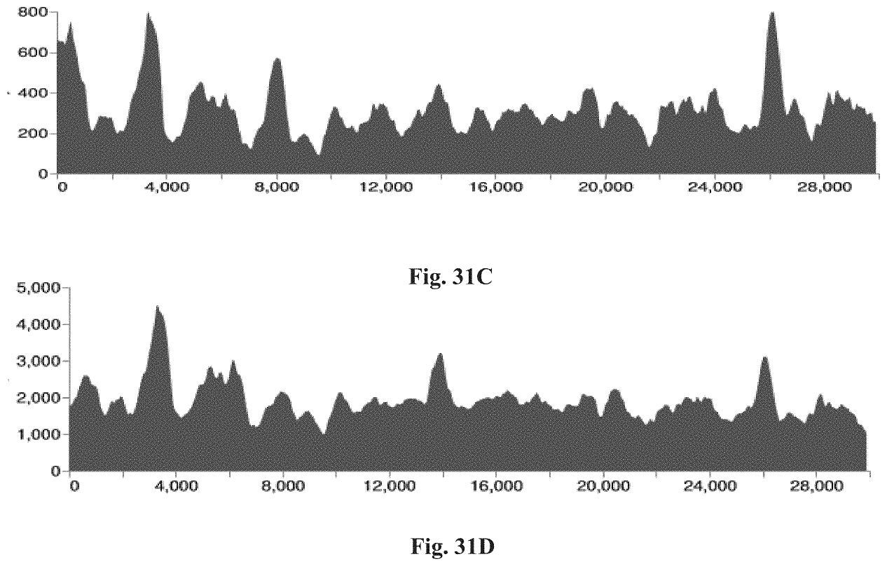

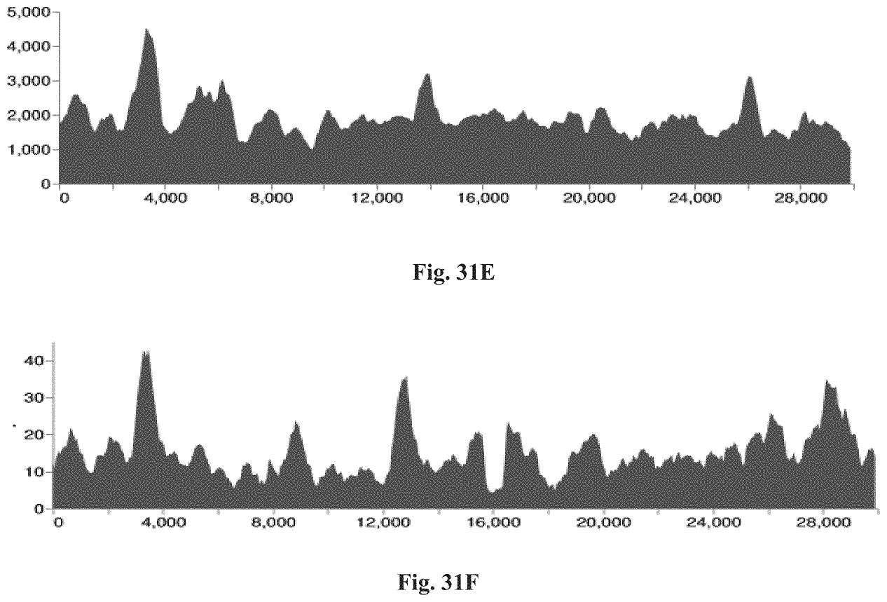

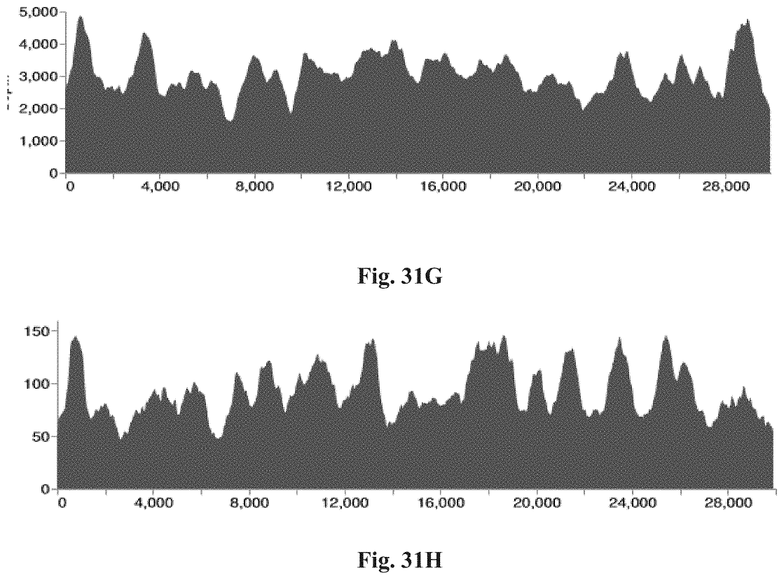

All fecal samples analyzed by enrichment NGS from positive patients by RT-PCR achieved 100% genome coverage of SARS-CoV-2 except for patient 3 which had 45%, and patient 10 which had 93% coverage (Table 25). The total number of SARS-CoV-2 mapped reads for patients 1, 3, 4, 6, 8, 10, 11, and 12 were 465645, 5984, 131582, 793603, 496852, 5929, 1270734, and 38256 respectively. The mean read depths of SARS-CoV-2 for patients 1, 3, 4, 6, 8, 10, 11, and 12 were 1129.8×, 31.7×, 318.6×, 1924.6×, 1206.7×, 15.5×, 3075.3×, and 92.7× respectively. The read depths at specific coordinates along the SARS-CoV-2 genome for each patient are captured in FIG. 31.

Table 25 documents the enrichment NGS metrics.

TABLE 25

| | Genome | Number of | Mapped | Mean |
|---|---|---|---|---|
| Sample ID | Coverage | Variants | Reads | Depth |
| Patient 1 | 100% | 11 | 465645 | 1129.8x |
| Patient 3 | 45% | 11 | 5984 | 31.7x |
| Patient 4 | 100% | 9 | 131582 | 318.6x |
| Patient 6 | 100% | 10 | 793603 | 1924.6x |
| Patient 8 | 100% | 10 | 496852 | 1206.7 |
| Patient 10 | 93% | 9 | 5929 | 15.6x |
| Patient 11 | 100% | 10 | 1270734 | 3075.3x |
| Patient 12 | 100% | 10 | 38256 | 92.7x |

Following alignment and mapping of SARS-CoV-2, patient genomes were compared to the Wuhan-Hu-1 (MN90847.3) SARS-CoV-2 reference genome via One Codex's bioinformatics pipeline to identify mutational variations. This analysis identified nucleotide variants at positions nt241 (C→T) and nt23403 (A→G) across all positive patients, and variants at positions nt3037 (C→T) and nt25563 (G→T) in seven of the eight patients (Table 3). Interestingly, patients 8, 11, and 12 harbored the same set of variants, as did patients 4 and 6 (who were kindreds). Unique variants not identified in any of the other individuals were detected in patients 1, 3, 6, and 10, with patient 3 harboring the most distinct SARS-CoV-2 genome with eight unique variants, followed by patient 1 with seven. Collectively, there were thirty-three different mutations among the patients in which SARS-CoV-2 was detected by whole genome enrichment NGS.

Table 26 documents the SARS-CoV-2 genomic positions, variant changes, and frequencies across the positive patient cohort.

TABLE 26

| | | | Patient | Patient | Patient | Patient | Patient | Patient | Patient | Patient |
|---|---|---|---|---|---|---|---|---|---|---|
| Region (ORF) | Position | Variant | 1 | 3 | 4 | 6 | 8 | 10 | 11 | 12 |
| 5′-UTR | 241 | C → T | 100% | 100% | 100% | 100% | 100% | 100% | 100% | 100% |
| 1a | 833 | T → C | x | x | x | x | 100% | x | 100% | 100% |
| 1a | 1059 | C → T | x | x | 100% | 100% | 99% | 100% | 100% | 100% |
| 1a | 1758 | C → T | x | x | 100% | 100% | x | x | x | x |
| 1a | 1973 | C → T | x | x | x | 87% | x | x | x | x |
| 1a | 3037 | C → T | 100% | x | 100% | 100% | 100% | 100% | 100% | 100% |
| 1a | 3078 | C → T | x | 89% | x | x | x | x | x | x |
| 1a | 4866 | G → T | 75% | x | x | x | x | x | x | x |
| 1a | 6720 | C → T | 93% | x | x | x | x | x | x | x |
| 1a | 8102 | G → T | x | 100% | x | x | x | x | x | x |
| 1a | 9401 | T → C | x | x | x | x | x | 64% | x | x |
| 1a | 9403 | T → A | x | x | x | x | x | 64% | x | x |
| 1a | 10870 | G → T | x | x | 100% | 100% | x | x | x | x |
| 1a | 11123 | G → A | x | x | 100% | 100% | x | x | x | x |
| 1b | 14408 | C → T | 100% | x | 100% | 100% | 100% | x | 100% | 100% |
| 1b | 14877 | C → T | x | 100% | x | x | x | x | x | x |
| 1b | 16616 | C → T | x | x | x | x | 100% | x | 100% | 100% |
| 1b | 16848 | C → T | 100% | x | x | x | x | x | x | x |
| 1b | 18652 | C → A | x | x | x | x | x | 83% | x | x |
| 1b | 19989 | T → G | x | 100% | x | x | x | x | x | x |
| Spike | 21576 | T → G | x | 83% | x | x | x | x | x | x |
| Spike | 23264 | G → A | x | 75% | x | x | x | x | x | x |
| Spike | 23403 | A → G | 100% | 100% | 100% | 100% | 100% | 100% | 100% | 100% |
| Spike | 23603 | C → T | 82% | x | x | x | x | x | x | x |
| 3a | 25563 | G → T | x | 100% | 100% | 100% | 100% | 100% | 100% | 100% |
| 3a | 25976 | C → A | x | x | x | x | 100% | x | 100% | 100% |
| 8 | 27964 | C → T | x | x | x | x | 100% | x | 100% | 100% |
| Nucleoprotein | 28881 | G → A | 100% | x | x | x | x | x | x | x |
| Nucleoprotein | 28882 | G → A | 100% | x | x | x | x | x | x | x |
| Nucleoprotein | 28883 | G → C | 100% | x | x | x | x | x | x | x |
| Nucleoprotein | 28997 | C → T | x | 100% | x | x | x | x | x | x |
| Nucleoprotein | 29019 | A → T | x | 100% | x | x | x | x | x | x |
| Nucleoprotein | 29364 | C → G | x | x | x | x | x | 85% | x | x |

Discussion: Coronaviridae is a family of enveloped, single-stranded, positive-sense RNA viruses. The total length of the genome is 30 Kb, consisting of a 5′-terminal noncoding region, an open reading frame (ORF) 1a/b-coding region, an S region encoding the spike glycoprotein (S protein), an E region encoding the envelope protein (E protein), an M region encoding the membrane protein (M protein), an N region encoding the nucleocapsid protein (N protein), and a -3′-terminal noncoding region. Among them, the poly protein encoded in the ORF1a/b region of the nonstructural protein can be cut by 3CLpro and PLpro of the virus to form RNA-dependent RNA polymerase and helicase, which guides the replication, transcription, and translation of the virus genome. The M and E proteins are involved in the formation of the envelope, while the N protein is involved in assembly. The spike protein binds to the receptor of the host cell and confers specificity for viral invasion into susceptible cells.

It is believed this is the first study to report whole genome sequencing (WGS) of SARS-CoV-2 from stool samples. The study was able to identify SARS-CoV-2 in patients that tested positive by nasopharyngeal swab RT-PCR analysis and observed unique genomes in 62.5% of the NGS positive patients. The overall homology among the genomes was high (99.97%), with variations identified in the ORF regions 1a, 1b, S, 3a, 8, and N. Of particular interest, was the adenine to guanine change in the S protein at position nt23403 which converts aspartic acid to glycine (D→G). The conversions of glycine to arginine (nt28883) and proline to arginine (nt29364) in the nucleoprotein are also of particular interest. While enrichment NGS is both costly and time consuming, these striking results highlight the potential viability of SARS-CoV-2 in feces, its possible role in transmission, and may accurately document complete eradication of the virus.

FIGS. 31A-31H are a series of graphs depicting whole genome alignment of SARS-CoV-2 in patients (Pt). The x-axis depicts the genomic coordinates as aligned to the MN908947.3 reference genome, and the y-axis represents the read depth at specific loci. FIG. 31A is patient 1. FIG. 31B is patient 3. FIG. 31C is patient 4. FIG. 31D is patient 6. FIG. 31E is patient 8. FIG. 31F is patient 10. FIG. 31G is patient 11. FIG. 31H is patient 12.

Conclusion: These results highlight the potential viability of SARS-CoV-2 in feces, its ongoing mutational accumulation, and its possible role in fecal-oral transmission. This study also elucidates the advantages of SARS-CoV-2 enrichment NGS, which may be a key methodology to document complete viral eradication.

Having thus described the invention, it should be apparent that numerous structural modifications and adaptations may be resorted to without departing from the scope and fair meaning of the instant invention as set forth herein above and described herein below by the claims.

1. A method of testing for specific organisms in an individual, the method comprising the steps of:
a) screening the individual;
b) acquiring a stool sample from the individual;
c) processing the stool sample to obtain the individual's microbiome, wherein the step of processing comprises the sub-steps of:
i) extracting DNA from the stool sample, which comprises adding the stool sample to a bead beating tube, achieving cell lysis, capturing the DNA on a silica membrane in a spin-column, and washing and eluting the DNA from the membrane; and
ii) purifying the extracted DNA;
d) sequencing the microbiome of the individual; and
e) analyzing the microbiome of the individual to determine whether one or more specific organisms are present in the individual, whereby a health condition of the individual is determined.

2. The method of claim 1, wherein step b) comprises providing the individual with a stool sample collection kit.

3. The method of claim 2, wherein the stool sample collection kit comprises:
a) at least one stool sample collection vial;
b) at least one toilet accessory;
c) at least one specimen bag;
d) at least one pair of gloves;
e) an authorization form;
f) a patient information card;
g) a questionnaire; and
h) stool sample collection instructions.

4. The method of claim 1, wherein step b) comprises acquiring the stool sample from the individual via colonoscopy.

5. The method of claim 1, wherein the one or more specific organisms of step e) comprise one or more of the following: Actinobacteria phylum, Acinetobacter baumannii, Actinomyces odontolyticus.

6. The method of claim 5, wherein the organism selected is Acinetobacter baumannii.

7. The method of claim 5, wherein the wherein the organism selected is Actinomyces odontolyticus.

8. The method of claim 5, wherein the wherein the wherein the organism selected is from the actinobacteria phylum.

9. (canceled)

10. The method of claim 1, wherein step e) is an assay that tests for the following organisms: Actinobacteria phylum, Acinetobacter baumannii, Actinomyces odontolyticus.

11. The method of claim 1, wherein step e) comprises comparing the microbiome of the individual to a microbiome of a mother of the individual.

12. The method of claim 1, wherein step e) comprises comparing the microbiome of the individual to a microbiome of a sibling of the individual.

13. The method of claim 1, wherein step e) comprises comparing the microbiome of the individual with a health condition to a microbiome of another individual with the same health condition.

14. The method of claim 1, wherein step e) comprises comparing the microbiome of the individual with a health condition to a microbiome of the individual before the individual had the health condition.

15. The method of claim 1, further comprising step f) after step e), storing the processed stool sample in a freezer.

16. A method of determining whether an individual has a health condition, the method comprising the steps of:
a) acquiring a stool sample from the individual;
b) processing the stool sample to obtain the individual's microbiome;
c) sequencing the microbiome of the individual; and
d) analyzing the microbiome of the individual to determine whether one or more specific organisms are present in the individual, whereby the health condition of the individual is determined.

17. The method of claim 16, wherein the health condition is selected from the group comprising: C. difficile infection, Obesity, Autism, Alzheimer's disease, Crohn's disease, Myalgic Encephalomyelitis/Chronic, Fatigue Syndrome (ME/CFS), Psoriasis, Chronic Urinary tract infection, Ulcerative Colitis, Multiple Sclerosis, Chronic constipation, Celiac sprue, Lyme disease, Elevated cholesterol, Colorectal cancer, Amyotrophic lateral sclerosis, Rheumatoid arthritis, Parkinson's disease, Depression, Anxiety, Obsessive-Compulsive disorder, Bipolar Disorder, Migraine headaches, Diabetes mellitus, Lupus, Epidermolysis, Metastatic mesothelioma, irritable bowel syndrome Diarrhea, irritable bowel syndrome Constipation, Eczema, Acne, Fatty liver, Myasthenia gravis, and Gout.

18. The method of claim 16, wherein step b) comprises the steps of:
i) extracting DNA from the sample, which comprises the steps of adding the stool sample to a bead beating tube, achieving cell lysis, capturing DNA on a silica membrane in a spin-column, and washing and eluting the captured DNA from the membrane; and
ii) purifying the extracted DNA.

19. The method of claim 16, wherein the one or more specific organisms of step d) are selected from the group consisting of: Actinobacteria phylum, Acinetobacter baumannii, Actinomyces odontolyticus.

20. A stool sample collection kit comprising:
a) at least one stool sample collection vial;
b) at least one toilet accessory;
c) at least one specimen bag;
d) at least one pair of gloves;
e) an authorization form;
f) a patient information card;
g) a questionnaire; and
h) stool sample collection instructions.
